(19) United States
(12) Patent Application Publication
(10) Pub. No.: US 2024/0248089 A1
(43) Pub. Date: Jul. 25, 2024

(54) METHOD AND DEVICE FOR DETECTING SIGLEC12

(71) Applicant: The Regents of the University of California, Oakland, CA (US)

(72) Inventors: Ajit Varki, La Jolla, CA (US); Nissi Varki, La Jolla, CA (US); Andrea Verhagen, La Jolla, CA (US); Shoib Siddiqui, La Jolla, CA (US)

(21) Appl. No.: 18/629,145

(22) Filed: Apr. 8, 2024

Related U.S. Application Data
(62) Division of application No. 16/552,876, filed on Aug. 27, 2019, now abandoned.
Application No. 16/552,876 is a continuation-in-part of application No. PCT/US2019/028341, filed on Apr. 19, 2019.
(60) Provisional application No. 62/659,884, filed on Apr. 19, 2018.
(60) Provisional application No. 62/723,858, filed on Aug. 28, 2018.

(51) Int. Cl. G01N 33/574; G01N 33/68

(52) U.S. Cl. CPC G01N 33/574; G01N 33/6893; G01N 2800/50; G01N 2800/52; G01N 2800/54

(57) ABSTRACT

The present application is in the field of sialic acid biochemistry, metabolism and antigenicity. More particularly, the present invention relates to the detection and analysis of Siglec-XII in a human biological sample for risk prediction, prognostication and diagnosis of disease. Also provided are devices configured to perform the methods disclosed herein.

### CROSS REFERENCE TO RELATED APPLICATION(S)

This application is a divisional of Ser. No. 16/552,876, filed Aug. 27, 2019, which claims the benefit of priority under 35 U.S.C. § 119(e) of U.S. Serial No. U.S. Ser. No. 62/723,858, filed Aug. 28, 2018, and is a continuation-in-part of International Application No. PCT/US2019/028341, filed Apr. 19, 2019, which claims the benefit of priority under 35 U.S.C. § 119(e) of U.S. Ser. No. 62/659,884, filed Apr. 19, 2018. The entire content of each of these applications is incorporated herein by reference.

### SEQUENCE LISTING

The instant application contains a Sequence Listing which has been submitted electronically in XML format and is hereby incorporated by reference in its entirety. Said XML copy, created on Apr. 5, 2024, is named 114198-5044_SL.xml and is 29 kilobytes in size.

### BACKGROUND OF THE INVENTION

### Field of the Invention

The invention relates generally to sialic acid biochemistry, and more specifically to the detection and analysis of Siglec-XII in a human biological sample for risk prediction, prognostication and diagnosis of disease.

### Background Information

All cells are covered with a dense and complex array of sugar chains. Sialic acids (Sias) are a family of nine-carbon sugars that are typically present at the outermost units of these sugar chains. By virtue of their terminal position, sialic acids act as binding sites for many exogenous and endogenous receptors such as the Influenza viruses and the Siglec family of endogenous proteins.

Siglecs (sialic acid binding Ig-like lectins) are immunoglobulin superfamily member lectins that selectively recognize different types and linkages of sialic acids, which are major components of cell surface and secreted glycoconjugates. The reported human Siglecs are type I membrane proteins, consisting of an amino-terminal Ig V-set domain, variable numbers of Ig C2-set domains, a single-pass transmembrane domain, and a cytoplasmic tail typically containing tyrosine-based signaling motifs. Sialic acid recognition is mediated by the first Ig V-set domain, and certain amino acid residues invariant to this domain are known to be involved in interactions with the sialic acid ligand. In particular, all Siglec V-set domains have a conserved arginine residue that forms a salt bridge with the carboxylate group of sialic acids. Experimental mutation of this residue markedly diminishes binding in all Siglecs studied to date.

Several studies have used monoclonal antibodies to detect various Sias in human tumors and tissues. Since its discovery almost 2 decades ago (1, 2) the human SIGLEC12 gene has not been a subject of much investigation. In fact, it has been largely ignored, because it encodes a protein (Siglec-XII) characterized by a human-specific universally-fixed mutation that eliminates a canonical functional feature of Siglecs (1), and also because it harbors a common polymorphic frameshift mutation that causes complete loss of protein expression in many humans (3). Despite such features suggesting an evolutionary loss of relevance to human biology, the locus appears to be undergoing selection favoring the null state (4). Earlier studies on a cohort of early stage prostate cancers showed no correlation between the frame-shift mutation and carcinoma risk (3). However, a Siglec-XII null tumor cell line transfected with SIGLEC12 cDNA showed more rapid growth in athymic mice (3). It is therefore hypothesized that while SIGLEC12 lost its original functions prior to the origin of humans, it may now affect the pathobiology of advanced carcinomas, which are very common in humans. Thus, a need exists for a simple test to detect and/or analyze this specific Siglec to determine risk, prognostication and diagnosis of disease.

### SUMMARY OF THE INVENTION

The present application is in the field of sialic acid biochemistry, metabolism and antigenicity. More particularly, the present invention relates to the detection and analysis of Siglec-XII in a human biological sample for risk prediction, prognostication and diagnosis of disease.

Accordingly, in one aspect, the invention provides a method for detecting the presence of wild type Siglec-XII in a subject. The method includes obtaining a sample containing epithelial cells from the subject; contacting the sample with a first monoclonal antibody that specifically binds to wild type Siglec-XII; and detecting the bound first monoclonal antibody, thereby detecting the presence of wild type Siglec-XII in the subject. In various embodiments the sample is urine or saliva. In various embodiments, the subject has cancer, such as skin cancer, colorectal cancer or prostate cancer. In various embodiments, the method may further include measuring the expression levels of one or more genes selected from the group consisting of IDO1, LCP1, BST2, CEACAM6, CXADR, TACSTD2, CTSF, and ZNF43, wherein elevated expression levels of any one or more of IDO1, LCP1, BST2, and CEACAM6, and wherein decreased expression levels of any one or more of CXADR, TACSTD2, CTSF, and ZNF43, as compared to expression levels in a corresponding normal sample indicates late stage progression of the cancer and/or risk of late stage progression of the cancer in the subject and a treatment for cancer should be initiated. In various embodiments, the method also includes administering a complex comprising the first monoclonal antibody and a toxin such as saporin, wherein the step of administering results in death of cells expressing wild type Siglec-XII, thereby treating the detected cancer in the subject. In various embodiments, wherein the toxin is conjugated to a second monoclonal antibody.

In another aspect, the invention provides a method for detecting cancer in a subject. The method includes obtaining a sample containing epithelial cells from the subject; contacting the sample with a first monoclonal antibody that specifically binds to wild type Siglec-XII; and detecting the bound first monoclonal antibody, thereby detecting the presence of wild type Siglec-XII in the subject. In various embodiments, the subject has cancer, such as skin cancer, colorectal cancer or prostate cancer. In various embodiments, the method also includes administering a complex comprising the first monoclonal antibody and a toxin such as saporin, wherein the step of administering results in death of cells expressing wild type Siglec-XII, thereby treating the detected cancer in the subject. In various embodiments, wherein the toxin is conjugated to a second monoclonal antibody.

In another aspect, the invention provides a method for detecting the severity of cancer in a subject undergoing treatment therefor. The method includes measuring the level of wild type Siglec-XII in a sample containing epithelial cells from the subject; and comparing the measured levels against reference levels obtained from a control subject. In various embodiments, the step of measuring comprises contacting the sample with a first monoclonal antibody that specifically binds to wild type Siglec-XII; and detecting the bound first monoclonal antibody, thereby detecting the presence of wild type Siglec-XII in the subject. In various embodiments, the presence of wild type Siglec-XII in the sample is indicative of late stage progression of the cancer in the subject and the treatment for cancer should be continued. In various embodiments, the sample is blood, urine or saliva. In various embodiments, the subject has cancer, such as colorectal cancer or prostate cancer. In various embodiments, the method further includes measuring the expression levels of one or more genes selected from the group consisting of IDO1, LCP1, BST2, CEACAM6, CXADR, TACSTD2, CTSF, and ZNF43, wherein elevated expression levels of any one or more of IDO1, LCP1, BST2, and CEACAM6, and wherein decreased expression levels of any one or more of CXADR, TACSTD2, CTSF, and ZNF43, as compared to expression levels in a corresponding normal sample indicates late stage progression of the cancer and/or risk of late stage progression of the cancer in the subject and the treatment for cancer should be continued. In various embodiments, the method also includes administering a complex comprising the first monoclonal antibody and a toxin such as saporin, wherein the step of administering results in death of cells expressing wild type Siglec-XII, thereby treating the detected cancer in the subject. In various embodiments, wherein the toxin is conjugated to a second monoclonal antibody.

In another aspect, the invention provides a method for predicting an adverse outcome in a subject undergoing a therapeutic regimen for cancer. The method includes measuring the level of wild type Siglec-XII in a first biological sample containing epithelial cells from the subject prior to beginning the therapeutic regimen; commencing the therapeutic regimen; and measuring the level of wild type Siglec-XII in a second biological sample from the subject obtained after commencing the therapeutic regimen. In various embodiments, the presence of wild type Siglec-XII in the second biological sample or an increased level of wild type Siglec-XII in the second biological sample as compared to the first biological sample is indicative of late stage progression of the cancer in the subject and the treatment for cancer should be continued. The method may further include measuring the levels of one or more genes selected from the group consisting of IDO1, LCP1, BST2, CEACAM6, CXADR, TACSTD2, CTSF, and ZNF43, wherein elevated levels of any one or more of IDO1, LCP1, BST2, and CEACAM6, and wherein decreased levels of any one or more of CXADR, TACSTD2, CTSF, and ZNF43, as compared to levels after therapy has commenced indicates late stage progression of the cancer and/or risk of late stage progression of the cancer in the subject and the treatment for cancer should be continued.

In another aspect, the invention provides a kit or article of manufacture comprising: (i) reagents specific to detect the presence and/or level of wild type Siglec-XII in a biological sample from a subject; and (ii) instructions for monitoring progression of cancer in the subject undergoing treatment for cancer or for predicting an adverse outcome or risk of an adverse outcome in a subject undergoing a therapeutic regimen for cancer. In various embodiments, the kit or article of manufacture also includes (iii) additional reagents specific to measure the levels of one or more of IDO1, LCP1, BST2, CEACAM6, CXADR, TACSTD2, CTSF, and ZNF43 in the biological sample; and (iv) additional instructions for monitoring progression of cancer in the subject undergoing treatment for cancer or for predicting an adverse outcome or risk of an adverse outcome in a subject undergoing a therapeutic regimen for cancer. In various embodiments, the kit includes a device for detecting the severity of cancer in a subject. The device includes a substrate having a surface, a monoclonal antibody that specifically binds to wild type Siglec-XII disposed on the surface of the substrate, and a detectable label that specifically binds to the monoclonal antibody.

### BRIEF DESCRIPTION OF THE DRAWINGS

FIGS. 1A-1D are pictorial diagrams showing enhanced expression of Siglec-XII in carcinomas. FIG. 1A shows expression of Siglec-XII studied in normal (benign) and cancer (malignant) human tissues using mouse monoclonal antibody clone 276. Representative examples of positive samples are shown. FIG. 1B shows the frequency of Siglec-XII detection on normal and cancer tissues (n=97 for normal tissues and n=85 for tumor samples, ***p value<0.001). FIG. 1C shows normal epithelium divided into squamous (n=35), columnar (n=14) and cuboidal (n=34). FIG. 1D shows carcinoma epithelium also divided into squamous (n=22), columnar (n=16) and cuboidal (n=32) (***p value<0.001).

FIGS. 2A-2F are graphical diagrams showing a correlation between SIGLECT12 genomic status and frequency or progression of late stage cancers. FIG. 2A shows prostate cancer patients diagnosed with PSA test followed up after 5 years. (NED-No evidence of disease: n=84, BCR—Biochemical Cancer Recurrence: n=28 and Met-Metastasis: n=10). FIG. 2B shows the Seventh-Day Adventist population where environmental risk factors for cancer are minimal. The percentage of patients with cancer and without cancer is shown to be either SIGLEC12−/− (non-expresser, n for cancer=14, n for non-cancer=20) or SIGLEC12+/− and SIGLEC12+/+ (expresser, n for cancer=40 and n for non-cancer-33). FIGS. 2C and 2D show the percentage of patients that are Siglec-XII expressers (SIGLEC12+/− and SIGLEC12+/+) or non-expressers (SIGLEC12−/−) in the FIRE3 and TRIBE stage IV colorectal cancer cohorts (FIRE3 cohort: expresser n=85, non-expresser n=16 and TRIBE cohort: expresser n=177, non-expresser n=27). FIGS. 2E and 2F show overall survival of colorectal cancer patients that are Siglec-XII expressers versus non-expressers (*p value<0.05).

FIGS. 3A-3C are graphical and pictorial diagrams showing Siglex-XII expression in association with cancer progression in advanced colorectal cancer cohort and correlation with overall survival. FIG. 3A shows and example of tissue sections with adjacent normal and malignant cells from a prostate cancer patient. FIG. 3B shows gene expression in Siglec-XII transfected prostate cancer cells versus sham transfection. Differentially expressed genes are highlighted, and genes not differentially expressed are shown in darker color. A fold change of 2 and p value<0.05 was used as a cut-off. FIG. 3C shows an exemplary heatmap showing the differentially expressed genes in the Siglec-XII expressing PC-3 cell line versus parental PC-3 cells (n=4).

FIGS. 4A-4E are pictorial and graphical diagrams showing the results from Population Genetic Analysis of SIGLEC12 locus and dot blot analysis of urinary epithelial cells to define Siglec-XII status. Signatures of selection in and around the SIGLEC12 locus. FIG. 4A shows signature of “Selective Sweep” in human population (EUR). The composite likelihood ratio (CLR) test of selective sweep based on the SFS is shown in blue. The black star indicates the location of frame-shift mutation (rs66949844). FIG. 4B shows an Estimation of Population differentiation “F_{ST}” (global) in three human populations (CHB_CEU_YRI). The bars show the relative value of F_{ST} (highest to lowest). FIG. 4C shows the results from Estimation of Tajima's D shows an excess of rare alleles and reduction in diversity in human populations (CHB, CEU and YRI).

FIG. 4D shows that urine and saliva samples were obtained from healthy individuals and used for checking protein expression of Siglec-XII by the dot blot. FIG. 4E shows that urine samples from multiple healthy donors were used to check protein expression of Siglec-XII. One typical example is shown.

FIGS. 5A and 5B are graphical diagrams showing targeting of carcinoma cells by Siglec-XII antibody. FIG. 5A shows antibody-mediated endocytosis of Siglec-XII resulted in MabZAP (goat anti-mouse toxin conjugate) internalization and resulted in reduced cell viability in Siglec-XII expressing PC-3 cells. FIG. 5B shows internalization of Siglec-XII in MDaPCa2b cells upon incubation with mAb276 for 2 h at 37° C. The closed square is the level of nonspecific staining of the cells treated with secondary antibody alone.

FIG. 6 shows human-specific changes in the SIGLEC12 gene. The alignment of exon 1 (nucleic acid residues 1 to 487 of SEQ ID NO: 2) of human and chimpanzee SIGLEC12 is shown. hSIGLEC12P is the human allele with an extra G, resulting in a frameshift. hSIGLEC12 is the wild type human allele. cSIGLEC12 is the chimpanzee SIGLEC12 allele. The signal peptide-coding region is underlined with a dashed line. The region with the extra G is underlined. The mutation from CGT (in chimp) to TGT (in humans) (boxed) results in an R122C mutation, which eliminates sialic acid binding. All of the human samples tested had the cysteine and thus do not bind sialic acid. hSIGLEC12P results in a premature termination codon, indicated by ***. Residues different from hSIGLEC12 have a gray background (with nucleic acid residues 24 to 29 of SEQ ID NO: 6 shown).

### DETAILED DESCRIPTION OF THE INVENTION

The present application is in the field of sialic acid biochemistry, metabolism and antigenicity. More particularly, the present invention relates to the detection and analysis of Siglec-XII in a human biological sample for risk prediction, prognostication and diagnosis of disease.

Before the present compositions and methods are described, it is to be understood that this invention is not limited to particular compositions, methods, and experimental conditions described, as such compositions, methods, and conditions may vary. It is also to be understood that the terminology used herein is for purposes of describing particular embodiments only, and is not intended to be limiting, since the scope of the present invention will be limited only in the appended claims.

As used in this specification and the appended claims, the singular forms “a”, “an”, and “the” include plural references unless the context clearly dictates otherwise. Thus, for example, references to “the method” includes one or more methods, and/or steps of the type described herein which will become apparent to those persons skilled in the art upon reading this disclosure and so forth.

It is intended that reference to a range of numbers disclosed herein (for example 1 to 10) also incorporates reference to all related numbers within that range (for example, 1, 1.1, 2, 3, 3.9, 4, 5, 6, 6.5, 7, 8, 9 and 10) and also any range of rational numbers within that range (for example 2 to 8, 1.5 to 5.5 and 3.1 to 4.7) and, therefore, all sub-ranges of all ranges expressly disclosed herein are expressly disclosed. These are only examples of what is specifically intended and all possible combinations of numerical values between the lowest value and the highest value enumerated are to be considered to be expressly stated in this application in a similar manner.

The term “comprising,” which is used interchangeably with “including,” “containing,” or “characterized by,” is inclusive or open-ended language and does not exclude additional, unrecited elements or method steps. The phrase “consisting of” excludes any element, step, or ingredient not specified in the claim. The phrase “consisting essentially of” limits the scope of a claim to the specified materials or steps and those that do not materially affect the basic and novel characteristics of the claimed invention. The present disclosure contemplates embodiments of the invention compositions and methods corresponding to the scope of each of these phrases. Thus, a composition or method comprising recited elements or steps contemplates particular embodiments in which the composition or method consists essentially of or consists of those elements or steps.

Unless defined otherwise, all technical and scientific terms used herein have the same meaning as commonly understood by one of ordinary skill in the art to which this invention belongs. Although any methods and materials similar or equivalent to those described herein can be used in the practice or testing of the invention, the preferred methods and materials are now described.

The term “cancer” as used herein, includes any cell having uncontrolled and/or abnormal rate of division that then invade and destroy the surrounding tissues. Cancer is a multistep process that can be defined in terms of stages of malignancy wherein the normal orderly progression is aberrant. In broad stages, normal tissue may begin to show signs of hyperplasia or show signs of neoplasia. As used herein, “hyperplasia” refers to cells that exhibit abnormal multiplication or abnormal arrangement in a tissue. Included in the term hyperplasia, are benign cellular proliferative disorders, including benign tumors. As used herein, “proliferating” and “proliferation” refer to cells undergoing mitosis. As used herein “neoplasia” refers to abnormal new growth, which results in a tumor. Unlike hyperplasia, neoplastic proliferation persists even in the absence of the original stimulus and characterized as uncontrolled and progressive. Malignant neoplasms, or malignant tumors, are distinguished from benign tumors in that the former show a greater degree of anaplasia and have the properties of invasion and metastasis. As used herein, “metastasis” refers to the distant spread of a malignant tumor from its sight of origin. Cancer cells may metastasize through the bloodstream, through the lymphatic system, across body cavities, or any combination thereof. Examples of cancer include but are not limited to, breast cancer, colon cancer, skin cancer, lung cancer, prostate cancer, hepatocellular cancer, gastric cancer, pancreatic cancer, cervical cancer, ovarian cancer, liver cancer, bladder cancer, cancer of the urinary tract, thyroid cancer, renal cancer, carcinoma, melanoma, head and neck cancer, and brain cancer. As used herein, squamous cell carcinoma (SCC) refers to cancer of squamous cells (keratinocytes), which are the main structural cells of the epidermis. Exemplary types of SCC include, but are not limited to, adenoid/pseudoglandular SCC, intraepidermal SCC, large cell keratinizing SCC, large cell non-keratinizing SCC, lymphoepithelial carcinoma, papillary SCC, papillary thyroid carcinoma, small cell keratinizing SCC, spindle cell SCC, and verrucous SCC. SCC of the skin, often referred to as cutaneous SCC, is usually found on areas of the body damaged by UV rays from the sun or tanning beds. Unlike other types of skin cancer, SCC can spread to the tissues, bones, and nearby lymph nodes, where it may become difficult to treat.

The term “subject” as used herein refers to any individual or patient to which the subject methods are performed. Generally, the subject is human, although as will be appreciated by those in the art, the subject may be an animal. Thus, other animals, including mammals such as rodents (including mice, rats, hamsters and guinea pigs), cats, dogs, rabbits, farm animals including cows, horses, goats, sheep, pigs, etc., and primates (including monkeys, chimpanzees, orangutans and gorillas) are included within the definition of subject.

As used herein, the terms “sample” and “biological sample” refer to any sample suitable for the methods provided by the present invention. In one embodiment, the biological sample of the present invention is a tissue sample, e.g., a biopsy specimen such as samples from needle biopsy (i.e., biopsy sample). In other embodiments, the biological sample of the present invention is a sample of bodily fluid, e.g., serum, plasma, sputum, lung aspirate, urine, and ejaculate.

Reference herein to “normal samples” or “corresponding normal samples” means biological samples of the same type as the biological sample obtained from the subject. In various embodiments, the corresponding normal sample is a sample obtained from a healthy individual. In various embodiments, the corresponding normal sample is a sample obtained from an otherwise healthy portion of tissue of the subject being tested for risk prediction, prognostication and diagnosis of disease. Such corresponding normal samples can, but need not, be obtained from an individual that is age-matched and/or of the same sex as the individual providing the sample being examined.

The term “therapeutically effective amount” or “effective amount” means the amount of a compound or pharmaceutical composition that will elicit the biological or medical response of a tissue, system, animal or human that is being sought by the researcher, veterinarian, medical doctor or other clinician. Thus, the term “therapeutically effective amount” is used herein to denote any amount of a formulation that causes a substantial improvement in a disease condition when administered to the patient as specified by previously determined clinical trials. The amount will vary with the condition being treated, the stage of advancement of the condition, and the type and concentration of formulation applied. Appropriate amounts in any given instance will be readily apparent to those skilled in the art or capable of determination by routine experimentation.

A “therapeutic effect,” as used herein, encompasses a therapeutic benefit and/or a prophylactic benefit as described herein.

The terms “administration” or “administering” are defined to include an act of providing a compound or pharmaceutical composition of the invention to a subject in need of treatment. The phrases “parenteral administration” and “administered parenterally” as used herein means modes of administration other than enteral and topical administration, usually orally or by injection, and includes, without limitation, intravenous and intramuscular injection.

As used herein, the terms “reduce” and “inhibit” are used together because it is recognized that, in some cases, a decrease can be reduced below the level of detection of a particular assay. As such, it may not always be clear whether the expression level or activity is “reduced” below a level of detection of an assay, or is completely “inhibited.” Nevertheless, it will be clearly determinable, following a treatment according to the present methods.

As used herein, “treatment” or “treating” means to administer a composition or drug to a subject or a system with an undesired condition. The condition can include a disease or disorder, such as cancer. “Prevention” or “preventing” means to administer a composition to a subject or a system at risk for the condition. The condition can include a predisposition to a disease or disorder. The effect of the administration of the composition to the subject (either treating and/or preventing) can be, but is not limited to, the cessation of one or more symptoms of the condition, a reduction or prevention of one or more symptoms of the condition, a reduction in the severity of the condition, the complete ablation of the condition, a stabilization or delay of the development or progression of a particular event or characteristic, or minimization of the chances that a particular event or characteristic will occur.

The terms “polypeptide,” “peptide” and “protein” are used interchangeably herein to refer to a polymer of amino acid residues. The terms apply to amino acid polymers in which one or more amino acid residue is an artificial chemical mimetic of a corresponding naturally occurring amino acid, as well as to naturally occurring amino acid polymers and non-naturally occurring amino acid polymers.

The term “amino acid” refers to naturally occurring and synthetic amino acids, as well as amino acid analogs and amino acid mimetics that function in a manner similar to the naturally occurring amino acids. Naturally occurring amino acids are those encoded by the genetic code, as well as those amino acids that are later modified, e.g., hydroxyproline, 7-carboxyglutamate, and O-phosphoserine. Amino acid analogs refers to compounds that have the same basic chemical structure as a naturally occurring amino acid, i.e., an a carbon that is bound to a hydrogen, a carboxyl group, an amino group, and an R group, e.g., homoserine, norleucine, methionine sulfoxide, methionine methyl sulfonium. Such analogs have modified R groups (e.g., norleucine) or modified peptide backbones, but retain the same basic chemical structure as a naturally occurring amino acid. Amino acid mimetics refers to chemical compounds that have a structure that is different from the general chemical structure of an amino acid, but that functions in a manner similar to a naturally occurring amino acid. Naturally encoded amino acids are the 20 common amino acids (alanine, arginine, asparagine, aspartic acid, cysteine, glutamine, glutamic acid, glycine, histidine, isoleucine, leucine, lysine, methionine, phenylalanine, proline, serine, threonine, tryptophan, tyrosine, and valine) and pyrrolysine and selenocysteine.

“Conservatively modified variants” applies to both amino acid and nucleic acid sequences. With respect to particular nucleic acid sequences, conservatively modified variants refers to those nucleic acids which encode identical or essentially identical amino acid sequences, or where the nucleic acid does not encode an amino acid sequence, to essentially identical sequences. Because of the degeneracy of the genetic code, a large number of functionally identical nucleic acids encode any given protein. For instance, the codons GCA, GCC, GCG and GCU all encode the amino acid alanine. Thus, at every position where an alanine is specified by a codon, the codon can be altered to any of the corresponding codons described without altering the encoded polypeptide. Such nucleic acid variations are “silent variations,” which are one species of conservatively modified variations. Every nucleic acid sequence herein which encodes a polypeptide also describes every possible silent variation of the nucleic acid. One of skill will recognize that each codon in a nucleic acid (except AUG, which is ordinarily the only codon for methionine, and TGG, which is ordinarily the only codon for tryptophan) can be modified to yield a functionally identical molecule. Accordingly, each silent variation of a nucleic acid that encodes a polypeptide is implicit in each described sequence.

As to amino acid sequences, one of skill will recognize that individual substitutions, deletions or additions to a nucleic acid, peptide, polypeptide, or protein sequence which alters, adds or deletes a single amino acid or a small percentage of amino acids in the encoded sequence is a “conservatively modified variant” where the alteration results in the substitution of an amino acid with a chemically similar amino acid. Conservative substitution tables providing functionally similar amino acids are well known in the art. Such conservatively modified variants are in addition to and do not exclude polymorphic variants, interspecies homologs, and alleles of the invention.

The term “antibody” as used herein refers to polyclonal and monoclonal antibodies and fragments thereof, and immunologic binding equivalents thereof. The term “antibody” refers to a homogeneous molecular entity, or a mixture such as a polyclonal serum product made up of a plurality of different molecular entities, and broadly encompasses naturally-occurring forms of antibodies (for example, IgG, IgA, IgM, IgE) and recombinant antibodies such as single-chain antibodies, chimeric and humanized antibodies and multi-specific antibodies. The term “antibody” also refers to fragments and derivatives of all of the foregoing, and may further comprise any modified or derivatised variants thereof that retains the ability to specifically bind an epitope. Antibody derivatives may comprise a protein or chemical moiety conjugated to an antibody. A monoclonal antibody is capable of selectively binding to a target antigen or epitope. Antibodies may include, but are not limited to polyclonal antibodies, monoclonal antibodies (mAbs), humanized or chimeric antibodies, camelized antibodies, single chain antibodies (scFvs), Fab fragments, F(ab′)2 fragments, disulfide-linked Fvs (sdFv) fragments, for example, as produced by a Fab expression library, anti-idiotypic (anti-Id) antibodies, intrabodies, nanobodies, synthetic antibodies, and epitope-binding fragments of any of the above.

Antibodies can be tested for anti-target polypeptide activity using a variety of methods well-known in the art. Various techniques may be used for screening to identify antibodies having the desired specificity, including various immunoassays, such as enzyme-linked immunosorbent assays (ELISAs), including direct and ligand-capture ELISAs, radioimmunoassays (RIAs), immunoblotting, and fluorescent activated cell sorting (FACS). Numerous protocols for competitive binding or immunoradiometric assays, using either polyclonal or monoclonal antibodies with established specificities, are well known in the art. Such immunoassays typically involve the measurement of complex formation between the target polypeptide and a specific antibody. A two-site, monoclonal-based immunoassay utilizing monoclonal antibodies reactive to two non-interfering epitopes on the target polypeptide is preferred, but other assays, such as a competitive binding assay, may also be employed. See, e.g., Maddox et al, 1983, J. Exp. Med. 158:1211, incorporated herein by reference.

The term “capture antibody” as used herein means an antibody which is typically immobilized on a solid support such as a plate, bead or tube, and which antibody binds to and captures analyte(s) of interest.

The term “detection antibody” as used herein means an antibody comprising a detectable label that binds to analyte(s) of interest. The label may be detected using routine detection means for a quantitative, semi-quantitative or qualitative measure of the analyte(s) of interest.

As used herein, the terms “manage”, “managing”, and “management” in the context of the administration of a therapy to a subject refer to the beneficial effects that a subject derives from a therapy (e.g., a prophylactic or therapeutic agent) or a combination of therapies, while not resulting in a cure of the disease or condition. In various examples, a subject is administered one or more therapies (e.g., one or more prophylactic or therapeutic agents) to “manage” the disease or condition so as to prevent the progression or worsening of the disease or condition.

As used herein, the term “marker” or “biomarker” in the context of an analyte means any antigen, molecule or other chemical or biological entity that is specifically found in circulation or associated with a particular tissue that it is desired to be identified in a biological sample or on a particular tissue affected by a disease or disorder, for example cancer.

CD33-related Siglecs (CD33rSiglecs) are a rapidly evolving gene family encoded by a subset of SIGLEC genes clustered on chromosome 19 in humans and chimpanzees, and for some there are no clear orthologs between human and mouse. CD33rSiglecs are homologous in sequence and typically expressed on immune cells. The C-terminal signaling domain in CD33-related Siglecs has an immunoreceptor tyrosine-based inhibitory motif (ITIM) or an immunoreceptor tyrosine-based switch motif. ITIMs typically recruit the protein tyrosine phosphatases SHP-1 and SHP-2 or the lipid phosphatase SHIP-1, generally resulting in inhibitory downstream signaling.

The primate SIGLEC12 gene encodes one of the CD33-related Siglec family of signaling molecules in immune cells. It has been previously reported that this gene harbors a human-specific missense mutation of the codon for an Arg residue required for sialic acid recognition. Recently, it has been shown that this R122C mutation of the Siglec-XII protein is a fixed missense mutation that eliminated the sialic acid binding property of this protein, a canonical functional feature of all other human Siglecs. Population analysis of the SIGLEC12 locus identified a polymorphic frameshift mutation, which leads to truncation of the Siglec-XII polypeptide, and loss of expression. The homozygous null state of this mutation is present in all human populations with an average frequency of ˜40%, and the locus was independently identified as undergoing an unexplained “negative selective sweep”, apparently favoring the null state.

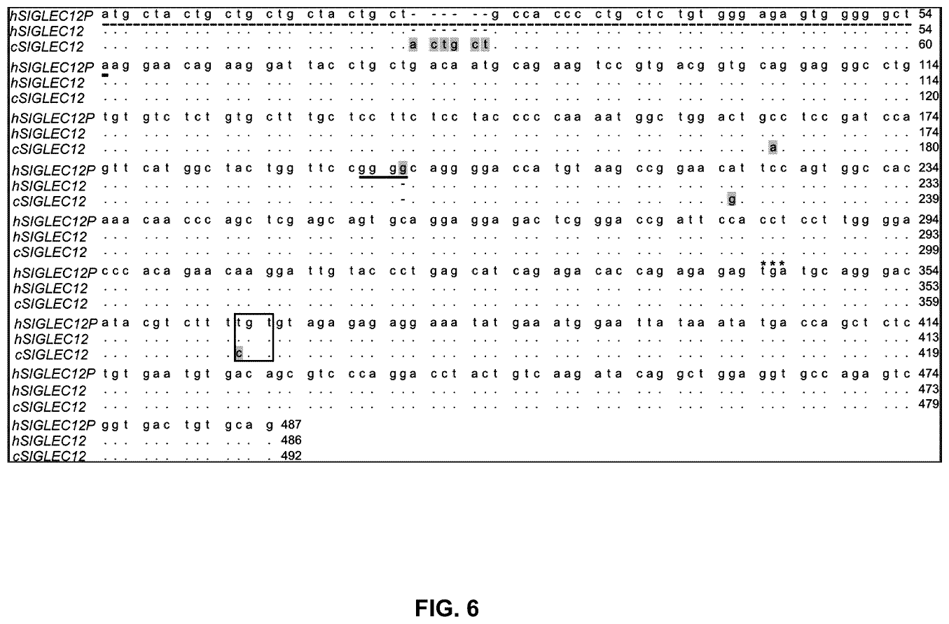

In addition to SIGLEC12, other Siglecs are also known to undergo human specific changes in sequence and expression (14). However, Siglec-XII (a receptor encoded by the SIGLEC12 gene) is often not even depicted in descriptions of human CD33rSiglecs, because of its inability to bind to Sia-bearing ligands, a canonical functional feature of these receptors. The R122C missense mutation universal to humans still leaves an open reading frame encoding a full-length Siglec-XII protein. As demonstrated herein, additional mutations in many humans have been found that would result in complete pseudogenization of SIGLEC12. As shown in FIG. 6, the most common mutation was a single nucleotide insertion in the first V-set exon, which if translated would result in a truncated protein of only 115 amino acids. This insertion mutation, a guanidine, occurs within a string of three other guanidines between base pairs 194 and 197 of the nucleotide sequence relative to the standard reference human genome sequence, so we cannot know precisely which guanidine is the actual insertion.

The most common inactivating mutation with a global allele frequency of 58% is a single nucleotide frameshift that markedly shortens the open reading frame. Unlike other CD33-related Siglecs that are primarily found on immune cells, it has been found that Siglec-XII protein is expressed not only on some macrophages but also on various epithelial cell surfaces in humans and chimpanzees. It has also been found that expression on certain human prostate epithelial carcinomas and carcinoma cell lines correlates with the presence of the non-frameshifted, intact SIGLEC12 allele. Although SIGLEC12 allele status did not predict prostate carcinoma incidence, restoration of expression in a prostate carcinoma cell line homozygous for the frameshift mutation induced altered regulation of several genes associated with carcinoma progression.

Thus, initial work was focused on a common polymorphic frame-shift mutation in human populations with an allele frequency ranging from 38% in sub-Saharan Africans to 86% in the Native American population (3). Another earlier study (4) suggested selection on SIGLEC12 based on the inactivating mutation rs16982743. However, another major frameshift mutation (rs66949844) was present in the human population at a frequency of ˜40%. Overall, this region of SIGLEC12 showed reduced genetic diversity and increased rare alleles, supported by a sweep scan showing a region of sweep in SIGLEC12. These findings were concordant with results from a study in six different human populations (32) showing a soft sweep in a region of SIGLEC12. The presence of excess rare alleles in and around a genomic region are also an indicator of a low level of population differentiation (33, 34) further indicating the presence of purifying selection or balancing selection. Purifying selection in SIGLEC12 region was also evident from the result of Tajima's D (TD) especially in the YRI population (African ancestry).

Previous studies showed that while the non-Sia binding Siglec-XII can be expressed in SIGLEC12 mutated PC-3 human prostate cancer cells, efforts to transfect the chimpanzee version of SIGLEC12 or the arginine restored version of human SIGLEC12 were not successful (3). This could be either due to rapid turnover or selection against expression in vitro. Regardless, the non-Sia-binding full-length human Siglec-XII is clearly different functionally, allowing persistent surface expression in malignant cells by as yet unknown mechanisms.

Analysis of chimpanzee, bonobo, gorilla, and orangutan sequences shows that they all have a functional SIGLEC12 gene with the key Arg residue intact. Because humans and chimpanzees are typically ˜99% identical in protein coding regions but have significant physiological, anatomical, and biomedical differences, it is important to explore these genetic variations. Remarkably, while cancers are common in humans, few are reported in chimpanzees, and are usually lymphomas or soft tissue tumors, unlike those that arise in humans, who are instead prone to epithelial carcinomas (19). Here immunohistochemistry analyses indicate that Siglec-XII is highly expressed in advanced carcinomas, as compared to normal epithelium. Considering the multiple mutations reported (3, 17) and others possible in the population, the overall expression in ˜35% in normal samples seems reasonable to represent the general mixed population. On the other hand, the high abundance of Siglec-XII in advanced carcinomas is remarkable, as is the high frequency of expression at >80%, in epithelial carcinomas.

While it has lost its Sia-binding property, Siglec-XII still has the ability to recruit SHP-1 and SHP-2 (2). SHP-2 (Tyrosine-protein phosphatase non-receptor type 11; PTPN-11) is a well-characterized oncogene that elicits cell growth, proliferation, tumorigenesis and metastasis (35). Over-activation and activating mutations of SHP-2 are known to be involved in breast cancer, leukemia and gliomas (35-37). Regardless, due to the role of SHP-2 in cancer progression there has been a thrust for developing specific inhibitors of the SHP-2 pathways. Sodium Stibogluconate (SSG) is an inhibitor of SHP-1 and SHP-2 that has been used recently in clinical trials for advanced solid tumors and melanoma (35). Elevated expression of Siglec-XII in tumors and association with SHP-2 may thus indicate cancer progression.

Moreover, the addition of SHP2 inhibitor to MEK inhibitors or ALK inhibitors has shown dramatic synergistic effects. Recent data has shown that SHP2 is intimately involved in the biology and progression of carcinomas, by linking cell surface receptors to oncogenic RAS-RAF-MEK-ERK pathways. While the fixed missense mutation in SIGLEC12 eliminates canonical sialic acid binding function, the protein still recruits SHP2, and accelerates tumor growth in a mouse model. We hypothesized that the dysfunctional Siglec-XII protein is involved in cancer progression in humans through aberrant recruitment of SHP2 in epithelia. Indeed, we found that expressing Siglec-XII in a carcinoma cell line enriched transcription of sets of genes associated with cancer progression, significantly correlating with tumorigenic signatures of available transcriptomic analysis of SHP2-modulated cancer cells. Interestingly, in this study we have found that a SHP2-binding receptor Siglec-XII to be highly upregulated in many carcinomas. These data indicate that the Siglec-XII-SHP2 axis may play a role in the human propensity for advanced carcinoma progression.

To explore molecular mechanisms of Siglec-XII in cancer progression, RNA expression patterns between PC-3 and PC-3-SiglecXII cell lines were compared. One of the top hits among the up-regulated genes in RNA-Seq was IDO-1, (Indoleamine 2,3-dioxygenase 1), an enzyme involved in conversion of tryptophan to kynurenine metabolites. This enzyme is highly up-regulated in many types of cancers. It is known that a decrease in the levels of tryptophan and an increase in the levels of kynurenine leads to immunosuppression and enhanced tumor growth (24, 38, 39). The molecular mechanisms for the effects of IDO-1 overexpression point towards maintenance of immunosuppression in tumor microenvironment, due to depletion of effector T cells and enrichment of regulatory T cells (24). There has been a recent focus on IDO-1 targeting through small molecule inhibitors in preclinical and clinical settings (38, 40). The top hit among the down-regulated genes was coxsackie and adenovirus receptor (CAR) which is a tight junction (Tj) protein mostly expressed on epithelial and endothelial cells (41, 42). It was identified as a cell surface receptor, which binds to coxsackie B viruses and adenoviruses. CAR literature related to tumor biology is confusing; for example, the role of CAR is dependent on cancer type and stage but in general it is considered a “tumor suppressor” gene (28). As such, the refined search performed by the inventors mainly focused on prostate cancer. In one study, CAR was shown to be down-regulated in prostate cancer cells (43) while in another an overexpression of CAR in PC-3 cell led to a decrease in the incidence of cancer and reduced tumor size (44). Down-regulation of CAR in PC-3-SigXII cells fits well with these findings.

Accordingly, population studies on four cancer cohorts were performed. The first was a prostate cancer cohort that was studied earlier (3). While a 5-year follow up for 122 patients was recorded in this cohort, there was still no observable positive correlation between SIGLEC12 pseudogenization and outcome. Without being bound by theory, one reason for this negative result may be that out of 122 patients only 10 developed metastasis (poor prognosis) and this might not be a sufficient number to find the relevance of SIGLEC12 in prognosis. The second cohort tested was a Seventh-day Adventist group and the lack of correlation could be due to two reasons. Firstly, most of the cancer patients in this group represented early stage cancer, where the effect of Siglec-XII is not pronounced. Secondly, many cancer risk factors such as intake of red meat, smoking, drinking alcohol etc., are minimal in this cohort, so it might be that Siglec-XII plays a role only when other obvious risk factors are involved.

In one population, it was discovered that the null state of a gene affects the prognosis of advanced carcinomas. Remarkably, this gene (SIGLEC12) is mostly considered as a “pseudogene” due to the presence of the arginine mutation in the human population. According to the well-established theoretical concept, natural selection occurs in pre-reproductive or reproductive individuals (7). However, humans are a rare species that have prolonged post-reproductive lifespan (PRLS), and according to the ‘grandmother hypothesis’ inclusive fitness of infertile elderly caregivers can determine the fate of helpless grandchildren (9, 45). Again, without being bound by theory, the negative selection against SIGLEC12 in human populations could be due to enrichment in late stage carcinomas that mostly occur in middle to late life. As such, the work described herein appears to be the first potential example of inclusive fitness effects selecting for cancer suppression, supporting a function for PRLS in humans. In contrast, an expansion in the number of p53 genes maybe providing late life protection against cancer risk in long-lived elephants (46). However, elephants do not have a PRLS, so the selection mechanism must be different.

This first study of an unusual phenomenon raises even more questions than answers. We do not know of a definite ligand for Siglec-XII. While it does not bind with Sias, its interaction with other unknown ligand(s) cannot rule out. Conversely, one may consider the hypothesis that this is a constitutively active receptor, which does not need any ligand for its activation. This aspect of Siglec biology is not extensively studied. Secondly, it has been observed that besides epithelium, Siglec-XII is also expressed on some tissue macrophages.

More extensive RNA Seq analysis is needed to pinpoint the pathways that were up-regulated with Siglec-XII expression in PC-3 cells. Follow up studies are needed to explore the function of Siglec-XII in cancer progression. Thirdly, we did not pinpoint downstream signaling that occurs upon activation of Siglec-XII. Fourthly, future studies must decipher how Siglec-XII governs the expression of other genes that are differentially expressed. Regardless, we have previously noted that triggering of endocytosis by antibodies against this receptor can deliver toxins into the cell (3). In analogy to the targeting of Siglec-3/CD33 human leukemias (47), a similar approach could be taken for treatment of late stage carcinomas. A simple urine screen should be of value in these and other clinical studies.

Meanwhile, gene expression data comparing a tumor cell line transfected with a human SIGLEC12 cDNA with sham-transfected cells shows upregulation of pathways associated with cancer progression. Taken together, all of these evolutionary clues and experimental findings suggest that while SIGLEC12 likely lost its function in the immune system of humans, its persistence in some humans could be associated with the pathobiology of late stage carcinomas. In this regard, the present invention is based on the advantageous expression of wild type Siglec-XII on urinary epithelial cells, for development of a simple urine test to check for the expression status of SIGLEC12. In various embodiments, this approach can be combined with future genomic analyses and signaling studies, to explore this unusual human specific evolution of an apparent evolutionary liability. In addition, early detection of disease can increase the chances of successful use of targeted delivery of a toxin into human carcinoma cells.

Previous studies have shown that Siglec-3, -5, and -9 undergo rapid internalization upon cross-linking with antibodies. If a toxin is attached to such antibodies, toxin internalization also occurs, and this results in cell death. The present invention builds upon this observation using a monoclonal antibody conjugated to a toxin, such as saporin. Saporin is a ribosome inactivating protein from the seeds of Saponaria officinalis. Internalization of the Siglec would also deliver the monoclonal antibody conjugated toxin complex into the cell.

Thus, the present invention provides a method of determining the presence of wild type (wt) Siglec-XII in a biological sample from a subject. The method includes obtaining a sample from the subject and probing (i.e., contacting) the sample with a monoclonal antibody specific for Siglec-XII to determine the presence of disease. Human samples can be obtained from any bodily fluid or tissue, such as urine, saliva, etc. In various embodiments, the method further includes subsequently administering a monoclonal antibody conjugated to a toxin for targeted delivery of the toxin thereto. In various embodiments, the method further includes probing the sample with a labeled secondary antibody that binds to the monoclonal antibody, and thereafter, detecting the presence of the labeled antibody to determine the presence of wt Siglec-XII in the biological sample. In various embodiments, the sample is a urine sample from a subject having been diagnosed with cancer.

Methods to measure gene expression products are well known to a skilled artisan. Such methods to measure gene expression products, e.g., protein level, include ELISA (enzyme linked immunosorbent assay), western blot, immunoprecipitation, and immunofluorescence using detection reagents such as an antibody or protein binding agents. Alternatively, a peptide can be detected in a biological sample from a subject by introducing into the sample a labeled anti-peptide antibody and other types of detection agent. For example, the antibody can be labeled with a detectable marker whose presence in the sample is detected by standard imaging techniques.

In another aspect, the invention provides methods for detecting the progression of cancer in a subject undergoing treatment for cancer. The method includes measuring the level wild type Siglec-XII in a sample containing epithelial cells, such as bladder epithelial cells, from the subject; and comparing the measured levels against reference levels obtained from a control subject. In various embodiments, the presence of wild type Siglec-XII in the sample is indicative of late stage progression of the cancer in the subject and the treatment for cancer should be continued and/or the subject should be administered an alternative therapeutic regimen for the cancer.

In another aspect, the invention provides methods for predicting an adverse outcome in a subject undergoing a therapeutic regimen for cancer. The method includes measuring the level of wild type Siglec-XII in a first biological sample containing epithelial cells, such as bladder epithelial cells, from the subject prior to beginning the therapeutic regimen; commencing the therapeutic regimen; and measuring the level of wild type Siglec-XII in a second biological sample from the subject obtained after a predetermined time after commencing the therapeutic regimen. In various embodiments, the presence of wild type Siglec-XII in the second biological sample or an increased level of wild type Siglec-XII in the second biological sample as compared to the first biological sample is indicative of late stage progression of the cancer in the subject and the treatment for cancer should be continued and/or the subject should be administered an alternative therapeutic regimen for the cancer.

In various embodiments, a subject having been diagnosed with cancer will provide a urine or sputum sample prior to initiating cancer therapy, and again after being on the cancer therapy after a predetermined amount of time. Exemplary predetermined amounts of time useful in the methods of the present invention include, but are not limited to, 1 month, 3 months, 6 months, 9 months, 1 year, 2 years, and 3 years, depending on the overall health of the subject.

As such, the invention also provides a diagnostic panel of biomarkers for the detection and analysis of Siglec-XII in a human biological sample for risk prediction, prognostication and diagnosis of disease. In various embodiments, the panel may be used for detection of wild type Siglec-XII alone or in combination with measuring levels of one or more of: IDO1 (Indoleamine 2,3-Dioxygenase 1); LCP1 (Lymphocyte Cytosolic Protein 1); BST2 (Bone Marrow Stromal Cell Antigen 2); CEACAM6 (Carcinoembryonic Antigen Related Cell Adhesion Molecule 6); CXAD®(Coxsackievirus and adenovirus receptor); TACSTD2 (Tumor Associated Calcium Signal Transducer 2); CTSF (Cathepsin F); and ZNF43 (Zinc Finger Protein 43). Such biomarkers would be measured at multiple time-points to determine the degree of change (if any) in response to drug therapy to monitor the severity of the cancer throughout the therapeutic regimen. The results of each biomarker, and a laboratory interpretation will be provided back to the medical practitioner to determine if the subject should discontinue the cancer therapy and/or discontinue the cancer therapy and initiate an alternative cancer therapy. Thus, any of the above-described methods may further include measuring the expression levels of one or more genes selected from the group consisting of IDO1, LCP1, BST2, CEACAM6, CXADR, TACSTD2, CTSF, and ZNF43.

In various embodiments, the presence of wild type Siglec-XII in combination with increased expression of one or more of IDO1; LCP1; BST2; and CEACAM6 is indicative of late stage progression of the cancer and/or risk of late stage progression of the cancer in the subject and the treatment for cancer should be continued and/or an alternative therapeutic regimen for cancer should be initiated. In various embodiments, the presence of wild type Siglec-XII in combination with decreased expression of one or more of CXADR; TACSTD2; CTSF; and ZNF43 is likewise indicative of late stage progression of the cancer and/or risk of late stage progression of the cancer in the subject and the treatment for cancer should be continued and/or an alternative therapeutic regimen for cancer should be initiated. It should be understood that detecting the presence of wild type Siglec-XII in combination with increased expression of any one or more of IDO1; LCP1; BST2; and CEACAM6, and decreased expression of any one or more of CXADR; TACSTD2; CTSF; and ZNF43 is also indicative of late stage progression of the cancer and/or risk of late stage progression of the cancer in the subject and the treatment for cancer should be continued and/or an alternative therapeutic regimen for cancer should be initiated.

The invention also provides a method of determining whether a subject is amenable to treatment with a therapeutic regimen for cancer. The method can be performed, for example, by detecting the presence or absence of wild type Siglec-XII alone or in combination with measuring the levels of one or more of IDO1; LCP1; BST2; CEACAM6; CXADR; TACSTD2; CTSF; and ZNF43 in a biological sample containing epithelial cells, such as bladder epithelial cells, of a subject to be treated, and determining whether the levels of the biomarkers are elevated or decreased as compared to the levels of a corresponding normal sample and/or as compared to levels in a sample after therapy has commenced. As indicated above, detection of elevated or abnormally elevated levels of any one or more of IDO1; LCP1; BST2; and CEACAM6, or decreased or abnormally decreased levels of any one or more of CXADR; TACSTD2; CTSF; and ZNF43, in combination with the presence of wild type Siglec-XII in the sample as compared to the levels in a corresponding normal sample, or as compared to levels after therapy has commenced indicates late stage progression of the cancer in the subject and the treatment for cancer should be continued and/or an alternative therapeutic regimen for cancer should be initiated.

The present invention also contemplates commercial kits and articles of manufacture specific for performing the assays and methods described herein. In various embodiments, the kit or article of manufacture includes: (i) reagents specific to detect the presence and/or level of wild type Siglec-XII in a biological sample obtained from a subject; and (ii) instructions for monitoring progression of cancer in the subject undergoing treatment for cancer or for predicting an adverse outcome or risk of an adverse outcome in a subject undergoing a therapeutic regimen for cancer. In various embodiments, the kit or article of manufacture also includes (iii) additional reagents specific to measure the levels of one or more of IDO1, LCP1, BST2, CEACAM6, CXADR, TACSTD2, CTSF, and ZNF43 in the biological sample; and (iv) additional instructions for monitoring progression of cancer in the subject undergoing treatment for cancer or for predicting an adverse outcome or risk of an adverse outcome in a subject undergoing a therapeutic regimen for cancer. In various embodiments, the kit includes a device having a substrate on which is disposed a monoclonal antibody that specifically binds to Siglec-XII. Such substrates and means for attachment are well known to those of skill in the art. For example, various types of cellulose or agarose columns or ELISA plates are readily available. Elution of bound antibodies after binding to either solid phase can be easily accomplished by washing the solid phase with the manufacturer's recommended elution buffer, such as low pH.

For an initial indication of the role of Siglec-XII in prostate cancer development, the inventors monitored the growth of human prostate cancer cells stably transfected with human SIGLEC12 or empty vector in nude mice. Cells expressing Siglec-XII showed a significant growth advantage over nonexpressing cells. This small growth difference over 70 days (time period of the mouse experiment) could become pertinent over many years, the usual time that it takes for a clinically significant prostate cancer to develop in humans. It is currently unknown whether this result extends to humans, but this is testable by association studies on large cohorts with known outcomes. Thus, it is contemplated that the present invention may be used to determine the risk of relapse of cancer in a subject having undergone treatment for cancer.

Amino acid and nucleic acid sequences for the human proteins described above are known in the art. See, for example, Genbank Accession No. AF277806.1, human sialic acid-binding immunoglobulin-like lectin-like long splice variant, which provides the amino acid sequence (SEQ ID NO: 1):

MLLLLLLLPPLLCGRVGAKEQKDYLLTMQKSVTVQEGLCVSVLCSFSYPQ
NGWTASDPVHGYWFRAGDHVSRNIPVATNNPARAVQEETRDRFHLLGDPQ
NKDCTLSIRDTRESDAGTYVFCVERGNMKWNYKYDQLSVNVTASQDLLSR
YRLEVPESVTVQEGLCVSVPCSVLYPHYNWTASSPVYGSWFKEGADIPWD
IPVATNTPSGKVQEDTHGRFLLLGDPQTNNCSLSIRDARKGDSGKYYFQV
ERGSRKWNYIYDKLSVHVTALTHMPTFSIPGTLESGHPRNLTCSVPWACE
QGTPPTITWMGASVSSLDPTITRSSMLSLIPQPQDHGTSLTCQVTLPGAG
VTMTRAVRLNISYPPQNLTMTVFQGDGTASTTLRNGSALSVLEGQSLHLV
CAVDSNPPARLSWTWGSLTLSPSQSSNLGVLELPRVHVKDEGEFTCRAQN
PLGSQHISLSLSLQNEYTGKMRPISGVTLGAFGGAGATALVFLYFCIIFV
VVRSCRKKSARPAVGVGDTGMEDANAVRGSASQGPLIESPADDSPPHHAP
PALATPSPEEGEIQYASLSFHKARPQYPQEQEAIGYEYSEINIPK

Genbank Accession No. AAK51234.1, human sialic acid-binding immunoglobulin-like lectin-like long splice variant, which provides the nucleic acid sequence (SEQ ID NO: 2):

ATGCTACTGCTGCTGCTACTGCTGCCACCCCTGCTCTGTGGGAGAGTGGG
GGCTAAGGAACAGAAGGATTACCTGCTGACAATGCAGAAGTCCGTGACGG
TGCAGGAGGGCCTGTGTGTCTCTGTGCTTTGCTCCTTCTCCTACCCCCAA
AATGGCTGGACTGCCTCCGATCCAGTTCATGGCTACTGGTTCCGGGCAGG
GGACCATGTAAGCCGGAACATTCCAGTGGCCACAAACAACCCAGCTCGAG
CAGTGCAGGAGGAGACTCGGGACCGATTCCACCTCCTTGGGGACCCACAG
AACAAGGATTGTACCCTGAGCATCAGAGACACCAGAGAGAGTGATGCAGG
GACATACGTCTTTTGTGTAGAGAGAGGAAATATGAAATGGAATTATAAAT
ATGACCAGCTCTCTGTGAATGTGACAGCGTCCCAGGACCTACTGTCAAGA
TACAGGCTGGAGGTGCCAGAGTCGGTGACTGTGCAGGAGGGTCTGTGTGT
CTCTGTGCCCTGCAGTGTCCTTTACCCCCATTACAACTGGACTGCCTCTA
GCCCTGTTTATGGATCCTGGTTCAAGGAAGGGGCCGATATACCATGGGAT
ATTCCAGTGGCCACAAACACCCCAAGTGGAAAAGTGCAAGAGGATACCCA
CGGTCGATTCCTCCTCCTTGGGGACCCACAGACCAACAACTGCTCCCTGA
GCATCAGAGATGCCAGGAAGGGGGATTCAGGGAAGTACTACTTCCAGGTG
GAGAGAGGAAGCAGGAAATGGAACTACATATATGACAAGCTCTCTGTGCA
TGTGACAGCCCTGACTCACATGCCCACCTTCTCCATCCCGGGGACCCTGG
AGTCTGGCCACCCCAGGAACCTGACCTGCTCTGTGCCCTGGGCCTGTGAA
CAGGGGACGCCCCCCACGATCACCTGGATGGGGGCCTCCGTGTCCTCCCT
GGACCCCACTATCACTCGCTCCTCGATGCTCAGCCTCATCCCACAGCCCC

Genbank Accession No. AF277806.1, human sialic acid-binding immunoglobulin-like lectin-like short splice variant, which provides the amino acid sequence (SEQ ID NO: 3):

MLLPLLWANEERDSGGWADPRFSTASQDLLSRYRLEVPESVTVQEGLCVS
VPCSVLYPHYNWTASSPVYGSWFKEGADIPWDIPVATNTPSGKVQEDTHG
RFLLLGDPQTNNCSLSIRDARKGDSGKYYFQVERGSRKWNYIYDKLSVHV
TALTHMPTFSIPGTLESGHPRNLTCSVPWACEQGTPPTITWMGASVSSLD
PTITRSSMLSLIPQPQDHGTSLTCQVTLPGAGVTMTRAVRLNISYPPQNL
TMTVFQGDGTASTTLRNGSALSVLEGQSLHLVCAVDSNPPARLSWTWGSL
TLSPSQSSNLGVLELPRVHVKDEGEFTCRAQNPLGSQHISLSLSLQNEYT
GKMRPISGVTLGAFGGAGATALVFLYFCIIFVVVRSCRKKSARPAVGVGD
TGMEDANAVRGSASQGPLIESPADDSPPHHAPPALATPSPEEGEIQYASL
SFHKARPQYPQEQEAIGYEYSEINIPK

Genbank Accession No. AAK51233.1, human sialic acid-binding immunoglobulin-like lectin-like short splice variant, which provides the nucleic acid sequence (SEQ ID NO: 4):

ATGCTGCTGCCCCTGCTATGGGCAAATGAAGAGAGGGACAGTGGGGGCTG
GGCTGACCCTCGTTTCTCCACAGCGTCCCAGGACCTACTGTCAAGATACA
GGCTGGAGGTGCCAGAGTCGGTGACTGTGCAGGAGGGTCTGTGTGTCTCT
GTGCCCTGCAGTGTCCTTTACCCCCATTACAACTGGACTGCCTCTAGCCC
TGTTTATGGATCCTGGTTCAAGGAAGGGGCCGATATACCATGGGATATTC
CAGTGGCCACAAACACCCCAAGTGGAAAAGTGCAAGAGGATACCCACGGT
CGATTCCTCCTCCTTGGGGACCCACAGACCAACAACTGCTCCCTGAGCAT
CAGAGATGCCAGGAAGGGGGATTCAGGGAAGTACTACTTCCAGGTGGAGA
GAGGAAGCAGGAAATGGAACTACATATATGACAAGCTCTCTGTGCATGTG
ACAGCCCTGACTCACATGCCCACCTTCTCCATCCCGGGGACCCTGGAGTC
TGGCCACCCCAGGAACCTGACCTGCTCTGTGCCCTGGGCCTGTGAACAGG
GGACGCCCCCCACGATCACCTGGATGGGGGCCTCCGTGTCCTCCCTGGAC
CCCACTATCACTCGCTCCTCGATGCTCAGCCTCATCCCACAGCCCCAGGA
CCATGGCACCAGCCTCACCTGTCAGGTGACCTTGCCTGGGGCCGGCGTGA
CCATGACCAGGGCTGTCCGACTCAACATATCCTATCCTCCTCAGAACTTG
ACCATGACTGTCTTCCAAGGAGATGGCACAGCATCCACAACCTTGAGGAA
TGGCTCGGCCCTTTCAGTCCTGGAGGGCCAGTCCCTGCACCTTGTCTGTG
CTGTCGACAGCAATCCCCCTGCCAGGCTGAGCTGGACCTGGGGGAGCCTG
ACCCTGAGCCCCTCACAGTCCTCGAACCTTGGGGTGCTGGAGCTGCCTCG
AGTGCATGTGAAGGATGAAGGGGAATTCACCTGCCGAGCTCAGAACCCTC

Accession No. Q95LH0-1, Pan troglodytes (Chimpanzee) sialic acid-binding immunoglobulin-like lectin 12, which provides the amino acid sequence (SEQ ID NO: 5):

MLLLLLLLLLPPLLCGRVGAKEQKDYLLTMQKSVTVQEGLCVSVLCSFSY
PQNGWTDSDPVHGYWFRAGDHVSRNVPVATNNPARAVQEETRDRFHLLGD
PQNKDCTLSIRDTRESDAGTYVFRVERGNMKWNYKYDQLSVNVTASQDLL
SRYRLEVPESVTVQEGLCVSVPCSVLYPHCNWTASSPVYGSWFKEGADIP
CDIPVATNTPSGKVQEDTQGRFLLLGDPQTNNCSLSIRDARKGDSGKYYF
QVERGSRKWNYIYDKLSVHVTALTHLPTFSIPGTLESGHPRNLTCSVPWA
CEQGTPPTITWMGASVSSLEPTISRSSMLSLIPKPQDHGTSLTCQVTLPG
AGVTTTRAVRLNISYPPQNLTMTVFQGDGTASTTLRNGSALSVLEGQSLH
LVCAVDSNPPARLSWTWGSLTLSPSQSSNLGVLELPRVHVKDEGEFTCRA
QNPLGSQHISLSLSLQNEYTGKMRPISGVTLGAVGGAGATALVFLSFCII
FVVVRSCRKKSARPAVGVGDTGMEDTNAVRGSASQGPLIESPADDSPPHH
APPALATPFPEEGEIQYASLSFHKARPQYPQEQEAIGYEYSEINILK

Accession No. AF293372.1, Pan troglodytes (Chimpanzee) sialic acid-binding lectin Siglec-LI, which provides the nucleic acid sequence (SEQ ID NO: 6):

ATGCTACTGCTGCTGCTACTGCTACTGCTGCCACCCCTGCTCTGTGGGAG
AGTGGGGGCTAAGGAACAGAAGGATTACCTGCTGACAATGCAGAAGTCCG
TGACGGTGCAGGAGGGCCTGTGTGTCTCTGTGCTTTGCTCCTTCTCCTAC
CCCCAAAATGGCTGGACTGACTCCGATCCAGTTCATGGCTACTGGTTCCG
GGCAGGGGACCATGTAAGCCGGAACGTTCCAGTGGCCACAAACAACCCAG
CTCGAGCAGTGCAGGAGGAGACTCGGGACCGATTCCACCTCCTTGGGGAC
CCACAGAACAAGGATTGTACCCTGAGCATCAGAGACACCAGAGAGAGTGA
TGCAGGGACATACGTCTTTCGTGTAGAGAGAGGAAATATGAAATGGAATT
ATAAATATGACCAGCTCTCTGTGAATGTGACAGCGTCCCAGGACCTACTG
TCAAGATACAGGCTGGAGGTGCCAGAGTCGGTGACTGTGCAGGAGGGTCT
GTGTGTCTCTGTGCCCTGCAGTGTCCTTTACCCCCATTGCAACTGGACTG
CCTCTAGCCCTGTTTATGGATCCTGGTTCAAGGAAGGGGCCGATATACCA
TGCGATATTCCAGTGGCCACAAACACCCCAAGTGGAAAAGTGCAAGAGGA
TACCCAGGGTCGATTCCTCCTCCTTGGGGACCCACAGACCAACAACTGCT
CCCTGAGCATCAGAGATGCCAGGAAGGGGGATTCAGGGAAGTACTACTTC
CAGGTGGAGAGAGGAAGCAGGAAATGGAACTACATATATGACAAGCTCTC
TGTGCATGTGACAGCCCTGACTCACTTGCCCACCTTCTCCATCCCGGGGA
CCCTGGAGTCTGGCCACCCCAGGAACCTGACCTGCTCTGTGCCCTGGGCC
TGTGAACAGGGGACGCCCCCCACAATCACCTGGATGGGGGCCTCCGTGTC
CTCCCTGGAACCCACTATCTCCCGCTCCTCGATGCTCAGCCTCATCCCAA

GenBank Accession No. AAA36081.1, human, indoleamine 2,3-dioxygenase 1, which provides the amino acid sequence (SEQ ID NO: 7):

MAHAMENSWTISKEYHIDEEVGFALPNPQENLPDFYNDWMFIAKHLPDLI
ESGQLRERVEKLNMLSIDHLTDHKSQRLARLVLGCITMAYVWGKGHGDVR
KVLPRNIAVPYCQLSKKLELPPILVYADCVLANWKKKDPNKPLTYENMDV
LFSFRDGDCSKGFFLVSLLVEIAAASAIKVIPTVFKAMQMQERDTLLKAL
LEIASCLEKALQVFHQIHDHVNPKAFFSVLRIYLSGWKGNPQLSDGLVYE
GFWEDPKEFAGGSAGQSSVFQCFDVLLGIQQTAGGGHAAQFLQDMRRYMP
PAHRNFLCSLESNPSVREFVLSKGDAGLREAYDACVKALVSLRSYHLQIV
TKYILIPASQQPKENKTSEDPSKLEAKGTGGTDLMNFLKTVRSTTEKSLL
KEG

Accession No. P13796, human Lymphocyte Cytosolic Protein 1, which provides the amino acid sequence (SEQ ID NO: 8):

MARGSVSDEEMMELREAFAKVDTDGNGYISFNELNDLFKAACLPLPGYRV
REITENLMATGDLDQDGRISFDEFIKIFHGLKSTDVAKTFRKAINKKEGI
CAIGGTSEQSSVGTQHSYSEEEKYAFVNWINKALENDPDCRHVIPMNPNT
NDLFNAVGDGIVLCKMINLSVPDTIDERTINKKKLTPFTIQENLNLALNS
ASAIGCHVVNIGAEDLKEGKPYLVLGLLWQVIKIGLFADIELSRNEALIA
LLREGESLEDLMKLSPEELLLRWANYHLENAGCNKIGNFSTDIKDSKAYY
HLLEQVAPKGDEEGVPAVVIDMSGLREKDDIQRAECMLQQAERLGCRQFV
TATDVVRGNPKLNLAFIANLFNRYPALHKPENQDIDWGALEGETREERTF
RNWMNSLGVNPRVNHLYSDLSDALVIFQLYEKIKVPVDWNRVNKPPYPKL
GGNMKKLENCNYAVELGKNQAKFSLVGIGGQDLNEGNRTLTLALIWQLMR
RYTLNILEEIGGGQKVNDDIIVNWVNETLREAKKSSSISSFKDPKISTSL
PVLDLIDAIQPGSINYDLLKTENLNDDEKLNNAKYAISMARKIGARVYAL
PEDLVEVNPKMVMTVFACLMGKGMKRV

Accession No. Q10589, human Bone Marrow Stromal Antigen 2, which provides the amino acid sequence (SEQ ID NO: 9):

MASTSYDYCRVPMEDGDKRCKLLLGIGILVLLIIVILGVPLIIFTIKANS
EACRDGLRAVMECRNVTHLLQQELTEAQKGFQDVEAQAATCNHTVMALMA
SLDAEKAQGQKKVEELEGEITTLNHKLQDASAEVERLRRENQVLSVRIAD
KKYYPSSQDSSSAAAPQLLIVLLGLSALLQ

Accession No. P13688, human Carcinoembryonic Antigen Related Cell Adhesion Molecule, which provides the nucleic acid sequence (SEQ ID NO: 10):

MGHLSAPLHRVRVPWQGLLLTASLLTFWNPPTTAQLTTESMPFNVAEGKE
VLLLVHNLPQQLFGYSWYKGERVDGNRQIVGYAIGTQQATPGPANSGRET
IYPNASLLIQNVTQNDTGFYTLQVIKSDLVNEEATGQFHVYPELPKPSIS
SNNSNPVEDKDAVAFTCEPETQDTTYLWWINNQSLPVSPRLQLSNGNRTL
TLLSVTRNDTGPYECEIQNPVSANRSDPVTLNVTYGPDTPTISPSDTYYR
PGANLSLSCYAASNPPAQYSWLINGTFQQSTQELFIPNITVNNSGSYTCH
ANNSVTGCNRTTVKTIIVTELSPVVAKPQIKASKTTVTGDKDSVNLTCST
NDTGISIRWFFKNQSLPSSERMKLSQGNTTLSINPVKREDAGTYWCEVFN
PISKNQSDPIMLNVNYNALPQENGLSPGAIAGIVIGVVALVALIAVALAC
FLHFGKTGRASDQRDLTEHKPSVSNHTQDHSNDPPNKMNEVTYSTLNFEA
QQPTQPTSASPSLTATEIIYSEVKKQ

Accession No. P78310, human Coxsackievirus and adenovirus receptor, which provides the nucleic acid sequence (SEQ ID NO: 11):

MALLLCFVLLCGVVDFARSLSITTPEEMIEKAKGETAYLPCKFTLSPEDQ
GPLDIEWLISPADNQKVDQVIILYSGDKIYDDYYPDLKGRVHFTSNDLKS
GDASINVTNLQLSDIGTYQCKVKKAPGVANKKIHLVVLVKPSGARCYVDG
SEEIGSDFKIKCEPKEGSLPLQYEWQKLSDSQKMPTSWLAEMTSSVISVK
NASSEYSGTYSCTVRNRVGSDQCLLRLNVVPPSNKAGLIAGAIIGTLLAL
ALIGLIIFCCRKKRREEKYEKEVHHDIREDVPPPKSRTSTARSYIGSNHS
SLGSMSPSNMEGYSKTQYNQVPSEDFERTPQSPTLPPAKVAAPNLSRMGA
IPVMIPAQSK DGSIV

Accession No. P09758, human Tumor Associated Calcium Signal Transducer 2, which provides the nucleic acid sequence (SEQ ID NO: 12):

MARGPGLAPPPLRLPLLLLVLAAVTGHTAAQDNCTCPTNKMTVCSPDGPG
GRCQCRALGSGMAVDCSTLTSKCLLLKARMSAPKNARTLVRPSEHALVDN
DGLYDPDCDPEGRFKARQCNQTSVCWCVNSVGVRRTDKGDLSLRCDELVR
THHILIDLRHRPTAGAFNHSDLDAELRRLFRERYRLHPKFVAAVHYEQPT
IQIELRQNTSQKAAGDVDIGDAAYYFERDIKGESLFQGRGGLDLRVRGEP
LQVERTLIYYLDEIPPKFSMKRLTAGLIAVIVVVVVALVAGMAVLVITNR
RKSGKYKKVEIKELGELRKEPSL

Accession No. Q9UBX1, human Cathepsin F, which provides the nucleic acid sequence (SEQ ID NO: 13):

MAPWLQLLSLLGLLPGAVAAPAQPRAASFQAWGPPSPELLAPTRFALEMF
NRGRAAGTRAVLGLVRGRVRRAGQGSLYSLEATLEEPPCNDPMVCRLPVS
KKTLLCSFQVLDELGRHVLLRKDCGPVDTKVPGAGEPKSAFTQGSAMISS
LSQNHPDNRNETFSSVISLLNEDPLSQDLPVKMASIFKNFVITYNRTYES
KEEARWRLSVFVNNMVRAQKIQALDRGTAQYGVTKFSDLTEEEFRTIYLN
TLLRKEPGNKMKQAKSVGDLAPPEWDWRSKGAVTKVKDQGMCGSCWAFSV
TGNVEGQWFLNQGTLLSLSEQELLDCDKMDKACMGGLPSNAYSAIKNLGG
LETEDDYSYQGHMQSCNFSAEKAKVYINDSVELSQNEQKLAAWLAKRGPI
SVAINAFGMQFYRHGISRPLRPLCSPWLIDHAVLLVGYGNRSDVPFWAIK
NSWGTDWGEKGYYYLHRGSGACGVNTMASSAVVD

Accession No. P17038, human Zinc Finger Protein 43, which provides the nucleic acid sequence (SEQ ID NO: 14):

MGPLTFMDVAIEFCLEEWQCLDIAQQNLYRNVMLENYRNLVFLGIAVSKP
DLITCLEQEKEPWEPMRRHEMVAKPPVMCSHFTQDFWPEQHIKDPFQKAT
LRRYKNCEHKNVHLKKDHKSVDECKVHRGGYNGFNQCLPATQSKIFLFDK
CVKAFHKFSNSNRHKISHTEKKLFKCKECGKSFCMLPHLAQHKIIHTRVN
FCKCEKCGKAFNCPSIITKHKRINTGEKPYTCEECGKVFNWSSRLTTHKK
NYTRYKLYKCEECGKAFNKSSILTTHKIIRTGEKFYKCKECAKAFNQSSN
LTEHKKIHPGEKPYKCEECGKAFNWPSTLTKHKRIHTGEKPYTCEECGKA
FNQFSNLTTHKRIHTAEKFYKCTECGEAFSRSSNLTKHKKIHTEKKPYKC
EECGKAFKWSSKLTEHKLTHTGEKPYKCEECGKAFNWPSTLTKHNRIHTG
EKPYKCEVCGKAFNQFSNLTTHKRIHTAEKPYKCEECGKAFSRSSNLTKH
KKIHIEKKPYKCEECGKAFKWSSKLTEHKITHTGEKPYKCEECGKAFNHF
SILTKHKRIHTGEKPYKCEECGKAFTQSSNLTTHKKIHTGEKFYKCEECG
KAFTQSSNLTTHKKIHTGGKPYKCEECGKAFNQFSTLTKHKIIHTEEKPY
KCEECGKAFKWSSTLTKHKIIHTGEKPYKCEECGKAFKLSSTLSTHKIIH
TGEKPYKCEKCGKAFNRSSNLIEHKKIHTGEQPYKCEECGKAFNYSSHLN
THKRIHTKEQPYKCKECGKAFNQYSNLTTHNKIHTGEKLYKPEDVTVILT
TPQTFSNIK

### Example 1

### Methods and Materials

Cell culture—All of the prostate cancer cell lines, PC-3, MDaPCa2b, and LnCAP, and breast cancer cell lines MDA-MB-231 and MCF-7 were obtained from ATCC and grown as directed.

Mouse Monoclonal Antibody against Human Siglec-XII—A fusion protein Siglec-XII-Fc including the first three Ig-like domains of human Siglec-XII and the human IgG Fc domain was prepared. The fusion protein was used to immunize mice to generate monoclonal antibodies (BD Pharmingen). Two final clones 1130 and 276 were obtained. Specificity was confirmed by lack of cross-reactivity with Siglec-7-Fc. Studies were done using a mixture of the two clones or clone 276 or 1130 alone.

Flow cytometry—The cells were stained with anti-Siglec-XII monoclonal antibody 1130 or 276 or a mixture of the two to probe for Siglec-XII expression. The cells were lifted using 10 mm EDTA and washed with 1% BSA-PBS. 500,000 cells were aliquoted and incubated with 1 μg of anti-Siglec-XII monoclonal antibody 1130 or 276 or a mixture of the two for 1 h on ice. The cells were washed with 1 ml of 1% BSA-PBS and incubated with 1:100 GAM-RPE (Caltag) for 30 min on ice in dark. The cells were washed and resuspended in 400 μl of 1% BSA-PBS and read on FACSCalibur flow cytometer using Cellquest. The data were analyzed using FlowJo.

Immunohistochemistry Studies—Multi-tissue array slides were obtained from US Biomax (Rockville, Maryland), which were completely anonymized and consisted of normal human and cancer tissues. The sections were de-paraffinized and blocked for endogenous biotin and peroxidase. The heat-induced epitope retrieval was performed with citrate buffer pH 6. A 5-step signal amplification method was used which includes application of monoclonal anti-mouse Siglec-XII antibody (clone 276), followed by biotinylated donkey anti-mouse, horseradish peroxidase (HRP), Streptavidin, followed by application of the enzyme biotinyl tyramide and then labeled Streptavidin. The AEC kit (Vector) was used as substrate, nuclear counterstain was with Mayer's hematoxylin, and the slides were aqueous mounted for digital photographs, taken using the Olympus BH2 microscope.

Buccal Swab—Healthy volunteers were recruited, and their buccal swab samples were used for DNA isolation. Before collection of the swab, the donors were asked to remove the mucous layer of their cheek by rubbing sterile gauze against it. Subsequently a sterile cotton tip was rubbed on the inner cheek cells for genomic DNA isolation. Genomic DNA was isolated using the ChargeSwitch Buccal Cell gDNA isolation kit (Invitrogen, Cat No.-CS11021) according to the manufacturer's instructions. The PCR amplification for SIGLEC12 gene was performed using the primers:

Forward:
(SEQ ID NO: 15)
5′-CAATGCAGAAGTCCGTGACGGTGCAGG-3′;
and
Reverse:
(SEQ ID NO: 16)
5′-AGGATCAGGAGGGGCATCCAAGGTGC-3′.

The Phusion High Fidelity Polymerase kit was used according to the manufacturer's instructions. The DNA amplicon was purified using QIAquick PCR purification kit (Qaigen, Cat no.-28106) and it was sent for sequencing at Eton Bio, San Diego, using the sequencing primer: 5′-CTCTCTCTGGTGTCTCTGATGC-3′ (reverse) (SEQ ID NO: 17).

Dot Blot Using Urinefrom Healthy Donors—Healthy donors donated 50 ml of first morning urine according to the approved study. The urine sample was centrifuged at room temperature for 10 min at 500×g. The supernatant was removed, and cell pellet re-suspended in 100 μl PBS. The sample was applied onto nitrocellulose membrane and immobilized by applying negative pressure. The membrane was blocked using 50% Licor solution (cat no-927-40000)+50% PBST (PBS+0.01% Tween). After blocking, primary anti-Siglec-XII antibody (clone 1130) was applied at a dilution of 1:100-1:500. The primary antibody dilution was performed in 90% Licor Solution+10% PBST and incubation was carried out for 1 hour at room temperature (RT). The membrane was then washed with 10 ml PBST 3 times for 5 min each. After washing, the membrane was incubated with anti-mouse-Licor-800 antibody at a dilution of 1:10000 in 90% Licor Solution+10% PBST. The secondary antibody incubation was performed for 1 hour at RT in dark. After incubation the membrane was washed with PBST 3 times for 5 min followed by two times with PBS for 5 min. The band on the membrane was visualized by using Licor fluorescence scanning machine.

SIGLEC12 Frameshift Mutation—The Seventh-day Adventist group is a diverse population group where the key carcinogenesis risk factors are less prevalent, such as consumption of red meat, alcohol and smoking. The genomic DNA was isolated from the peripheral blood cells of 53 cancer patients and 54 age-matched control subjects. The frame-shift deletion mutation of SIGLEC12 was analyzed by first PCR amplifying the SIGLEC12 locus using the primers:

(forward)
(SEQ ID NO: 18)
5′-ACCCCTGCTCTGTGGGAGAGT-3′;
and
(reverse)
(SEQ ID NO: 19)
5′-AGGATCAGGAGGGGCATCCAAGGTGC-3′.

The PCR was performed using Phusion High Fidelity Polymerase kit. The amplified product was purified using the QIAquick PCR purification kit (Qaigen, cat no.-28106) and sent for sequencing to EtonBio, San Diego, USA. The sequencing was performed using the primer: 5′-CTCTCTCTGGTGTCTCTGATGC-3′ (reverse) (SEQ ID NO: 20).

RNA-Sequence Analysis—PC-3 and PC-3-SigXII expressing cells were cultured to confluency in T25 flasks and mRNA was extracted from the cells using the Qaigen RNeasy plus mini kit extraction mini-elute kit (Cat no.—74134). Transcriptomic analysis was performed on RNA libraries prepared from SIGLEC12 and control PC3 cells using the TruSeq RNA Library Prep Kit v2. Each cell line was used to prepare four separate technical replicate libraries for sequencing. Libraries were sequenced at 1×50 bp on HiSeq 4000 (Illumina). Reads were mapped to human reference genome Hg19 using STAR v2.5.3a (48). Mapped reads were counted at the gene level using featureCounts v1.5.2 (49) and counts were analyzed using DESeq2 v1.14.1 (50). Differentially expressed genes with a p-value ≤0.05 and fold change ≥2 were then selected for further examination and gene ontology term enrichment using PANTHER® database (51).

Statistical Analysis—Graph prism pad 5.0 was used. The chi-square test was performed on immunohistochemistry data, different cancer cohorts and a p value <0.05 was considered as significant. For the RNA-Seq the two-way ANOVA was used as the statistically significant value. The p value<0.05 and fold change of 2 was used as a cut-off for assessing the differentially expressed genes.

Population Genetics Analysis—Human genomes were accessed from the 1000 Genomes Project server (1000genomes.org/). Bed coordinates defining the SIGLEC12 genomic regions were retrieved from build hg19 using the University of California, Santa Cruz (UCSC), genome browser. A region containing SIGLEC12 gene was retrieved from the 1000 Genomes Project database and patterns of polymorphism were analyzed for each population (CHB, CEU and YRI), using the selection tools pipeline. Statistical tests such as frequency-based method (Tajima's D) and population differentiation-based methods (FST) among three different populations were analyzed (52). Each test is suited to detect selection at different timescales. Tajima's D is a commonly used summary of the site-frequency spectrum (SFS) of nucleotide polymorphism data and is based on the difference between two estimators of 0 (the population mutation rate 4Neμ): nucleotide diversity that is the average number of pairwise differences between sequences, and Watterson's estimator, based on the number of segregating sites. A negative Tajima's D signifies an excess of low frequency polymorphisms, and indicates a population size expansion, selective sweep, and/or positive selection, or negative selection. A positive Tajima's D value indicates a decrease in population size and/or that balancing selection (53). On the other hand, the estimator of population differentiation (FST), compares the variance of allele frequencies within and between populations (54). While large values of FST at a locus indicate complete differentiation between populations, which suggests directional selection, small values indicate the lack of differentiation in populations being compared, which might be an indicator of directional or balancing selection in both (55). Human genome raw data for SIGLEC12 (56) was utilized for detecting Selective Sweep using SweepFinder2 (57) which implements a composite likelihood ratio (CLR) test (58). The CLR uses the variation of the SFS of a region to compute the ratio of the likelihood of a selective sweep at a given position to the likelihood of a null model without a selective sweep. Outputs from selection Tools and Sweep scans were visualized in R using Plotly and examined for evidence of deviation from the null expectation.

### Example 2

### Enhanced Expression and Unexpectedly High Frequency of Siglec-XII in Carcinomas

Establishing Stable Transfectants of Prostate Carcinoma Cells Expressing Siglec-XII—PC-3 cells were transfected with PvuI linearized hSIGLEC12-pcDNA3.1(−) or empty pcDNA3.1(−) in six-well plates using Lipofectamine 2000 (Invitrogen). 48 h after transfection, the cells were trypsinized and grown with 800 μg/ml G418. After growing ˜1 month, expression of Siglec-12 was determined by flow cytometry. Four independent cell lines from four independent transfections were obtained: two with hSIGLEC12 and two with empty pcDNA 3.1(−).

Microarray Gene Expression Profile Comparison of Transfected and Sham-transfected Prostate Carcinoma Cells—RNA was isolated from the stably transfected PC-3 cell lines using the RNeasy mini kit (Qiagen). cDNA was synthesized and hybridized to Genechip Human Genome U133 Plus 2.0 Array (Affymetrix). Quantity and quality of final total RNA were examined using a nanodrop and with the RNA QC-Standard Bioanalyzer (Agilent). 5 μg of total RNA was used for cDNA synthesis, followed by in vitro transcription to incorporate biotin labels, and subsequent hybridization to Genechip human genome U133 Plus 2.0 array (Affymetrix) was performed by the GeneChip Microarray Core (University of California at San Diego) as described in the Affymetrix GeneChip protocol. The U133 Plus 2.0 interrogates ˜54,000 probe identification codes. The raw expression values were normalized using DNA chip analyzer, built May 8, 2008 (dChip), which is a Windows software package for probe level analysis of gene expression microarrays. Before further processing, the transcripts were filtered to 32,000 transcripts using the standard deviation for discrimination. The data were analyzed using rank products implemented within the Bioconductor project and the R program software (R is available as free software under the terms of the Free Software Foundation GNU General Public License). Heat maps were done using the dChip software. Functional analysis of genes was done using Ingenuity Pathways Analysis from Ingenuity Systems, Inc.

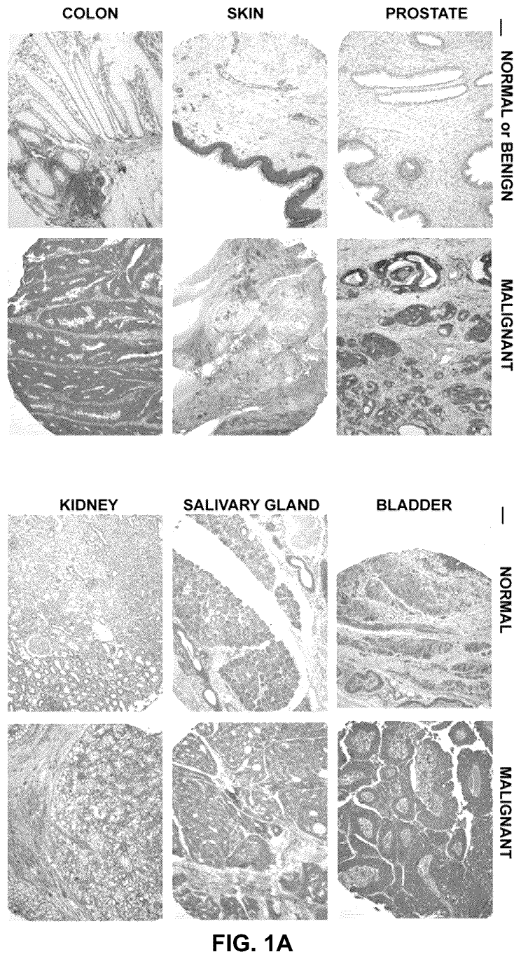

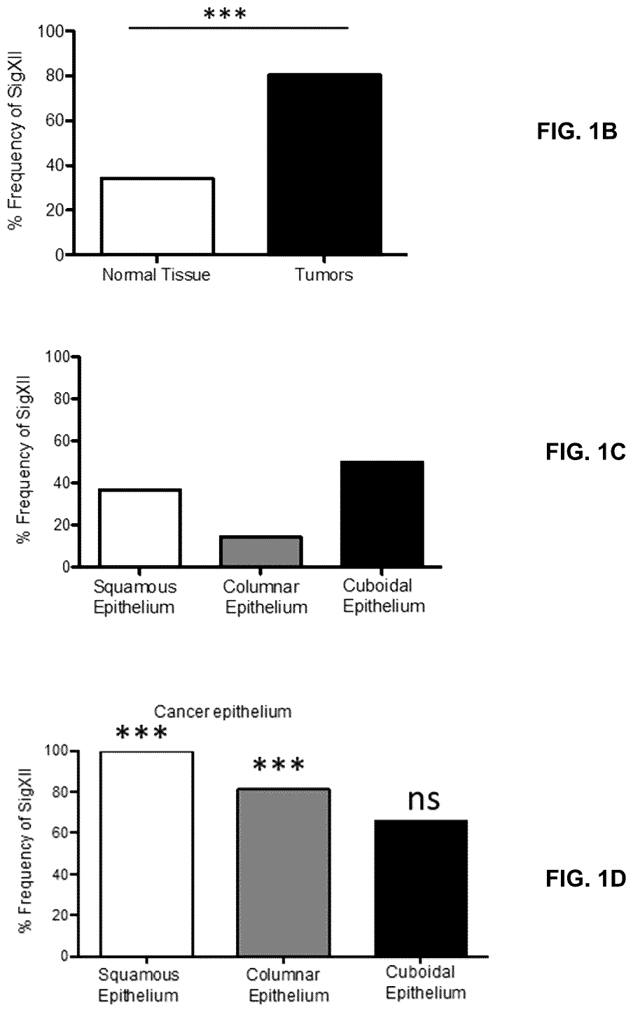

Immunohistochemical analyses for Siglec-XII showed low to moderate level expression in normal epithelia in a commercially available normal multi-tissue array with sample positivity of ˜35%. As many humans harbor a homozygous null state, this low frequency is not unexpected. In contrast, in a multi-tissue array with multiple malignancies, an abundance of expression was found in carcinomas (malignancies arising from epithelia) (FIG. 1A), with a much higher than expected frequency of Siglec-XII in cancers (˜80%) as compared to normal tissue (FIGS. 1B and 1C). Remarkably, 100% of the squamous cell carcinomas were positive (FIG. 1D). This result was obtained from a mixed population group aged between 21-75 years. Given the collection methods involved, the carcinomas are all likely in an advanced stage, and the implication is that individuals who can express Siglec-XII may be at the highest risk for dying with advanced carcinomas.

### Example 3

### No Correlation Between SIGLEC12 Genomic Status and Frequency or Progression of Early Stage Cancers

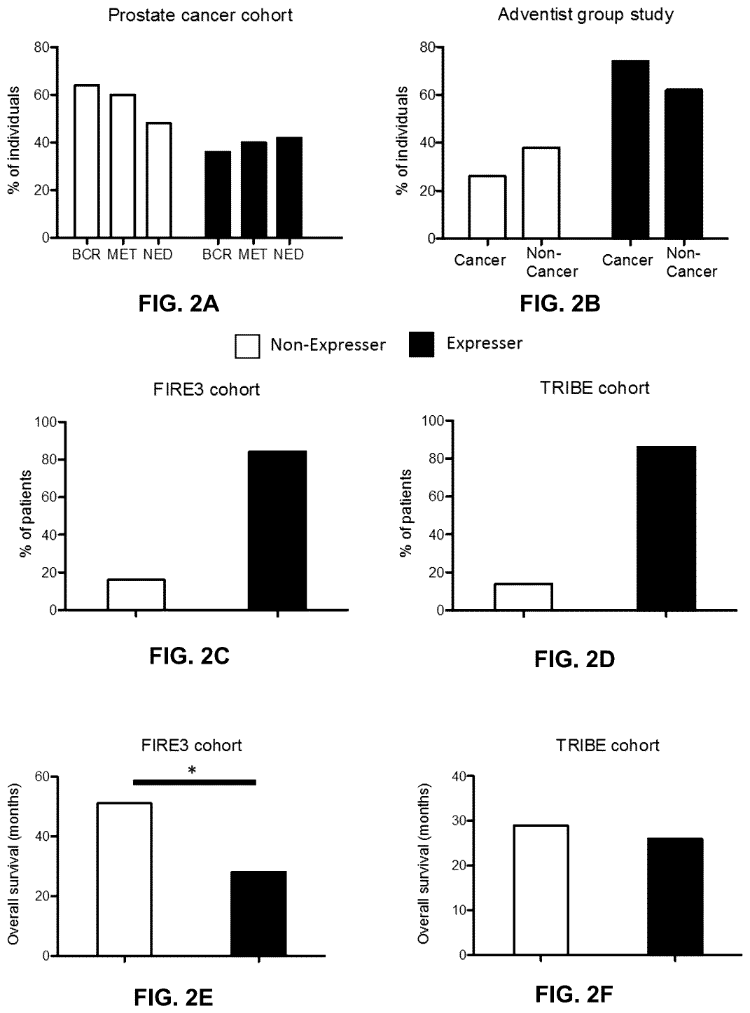

Next, it was determined if Siglec-XII expression predicts early carcinoma risk or progression in well-defined populations. It has been previously reported that the incidence of prostate cancer was not different between men with different SIGLEC12 genotypes (3). There was a minimum of 5-year follow up available for many of these patients categorized into no evidence of disease (NED); Biochemical recurrence (BCR) and Metastasis (Met). There was again no clear correlation of SIGLEC12 status with progression of these early stage carcinomas (FIG. 2A). Of course, most of these cases were originally diagnosed by measuring prostate specific antigen (PSA), which picks up many early stage cases that never progress in the lifetime of the individual (20). Indeed, if the patients with a poor outcome were compared to those with no evidence of disease recurrence following prostatectomy (NED), it was observed that most of the patients (84 out of 122) detected by PSA screening did not have disease progression at the time of follow up.

Seventh-day Adventists are members of a religious sect that do not smoke or consume alcohol, and have a largely vegetarian diet with limited intake of red meat (21). As usual risk factors for cancer are limited, carcinoma incidence is much lower than in the general population. The common frameshift insertion mutation (3) was genotyped on genomic DNA from the peripheral blood cells of 54 Seventh-day Adventist cancer patients and 53 non-cancer patients (age and sex-matched). Based on the initial findings in advanced carcinomas, it was hypothesized that Siglec-XII expressers may be more prone to develop carcinomas. However, while more Siglec-XII expressers were found in the cancer group, this trend supporting the hypothesis was not statistically significant (FIG. 2B). Notably, many of these cancers were diagnosed at an early stage. Taken together, the data suggest that the genomic status of SIGLEC12 may not be correlated with the early cancer risk.

### Example 4

High Frequency of SIGLEC12 Expression in Advanced Colorectal Cancer and Correlation with Overall Survival

Given the lack of significant correlation between SIGLEC12 status and carcinoma risk or early stage carcinomas, it was reasoned that there might instead be a correlation with late stage cancers. Indeed, in two stage IV colorectal cancer cohorts FIRE3 (592 patients from Germany and Austria) (22) and TRIBE (508 patients from Italy) (23) >80% of patients expressed SIGLEC12 based on the frameshift mutation (FIGS. 2C and 2D). This recapitulates the initial immunohistochemistry-based findings. Looking to see if SIGLEC12 status has prognostic value, overall survival in relation to Siglec-XII expression was checked. Interestingly, in FIRE3 the overall survival increased from 28 months to 51 months between Siglec-XII expressers and non-expressers (FIGS. 2E and 2F), i.e., a correlation between Siglec-XII expression and poor prognosis in late stage colorectal cancer.

### Example 5

Expression of Genes Associated with Cancer Progression in Siglec-XII Expressing Prostate Cancer Cell Line

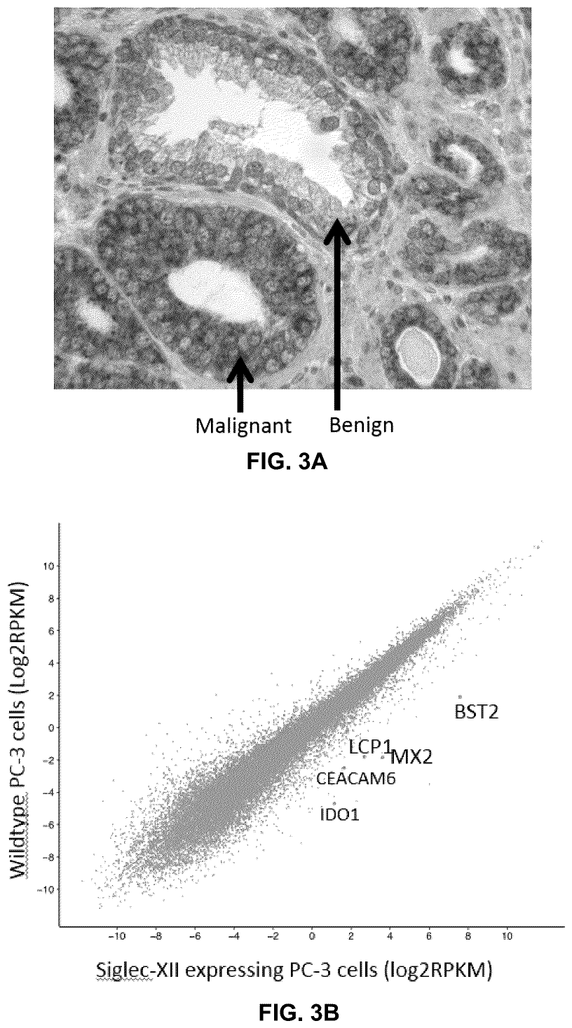

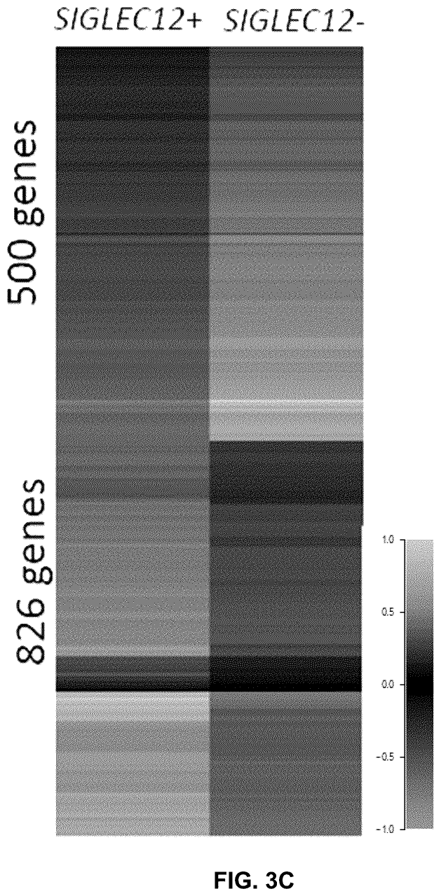

In keeping with this data supporting the relevance of Siglec-XII expression in advanced cancer, it was also noted that in tissue sections where both malignant and adjacent normal tissue were present, Siglec-XII expression was higher in the malignant cells (one such example is shown in FIG. 3A). To begin to explore the mechanism of action of this cell surface receptor, the inventors took advantage of an earlier model system in which the non-expressing PC-3 prostate carcinoma cells were transfected with a vector causing expression of Siglec-XII.

Larger tumors have already been observed when this PC-3-SiglecXII cell line was injected subcutaneously in the flanks of athymic nude mice, as compared to PC-3 cells transfected with vector alone (3). A one-tailed t test comparing the tumor volumes of SIGLEC12 PC-3 to pcDNA PC-3 tumors within each mouse showed a significant difference in mean size in four of five mice at p<0.05 (p=0.0492, 0.036, 0.0196, and 0.0134, respectively). Thus, the presence of the Siglec-XII resulted in significant increase in tumor volume.

Now, the RNA expression profiles were compared between these two cell lines and it was found that many genes were differentially expressed (FIGS. 3B and 3C). Importantly, these differentially expressed genes were enriched for genes known to play a role in cancer biology. A few of those up-regulated were IDO1 (Indoleamine 2,3-Dioxygenase 1) (24); LCP1 (Lymphocyte Cytosolic Protein 1) (25); BST2 (Bone Marrow Stromal Cell Antigen 2) (26); and CEACAM6 (Carcinoembryonic Antigen Related Cell Adhesion Molecule 6) (27), which are all involved in cancer progression. Among the down-regulated genes related to cancer progression were CXAD® (Coxsackievirus and adenovirus receptor) (28); TACSTD2 (Tumor Associated Calcium Signal Transducer 2) (29); CTSF (Cathepsin F) (30); and, ZNF43 (Zinc Finger Protein 43) (31) (FIG. 3A). Taken together these data support the notion that Siglec-XII expression facilitates late stage carcinoma progression.

### Example 6

### Further Evidence for Selection of the SIGLEC12 Locus

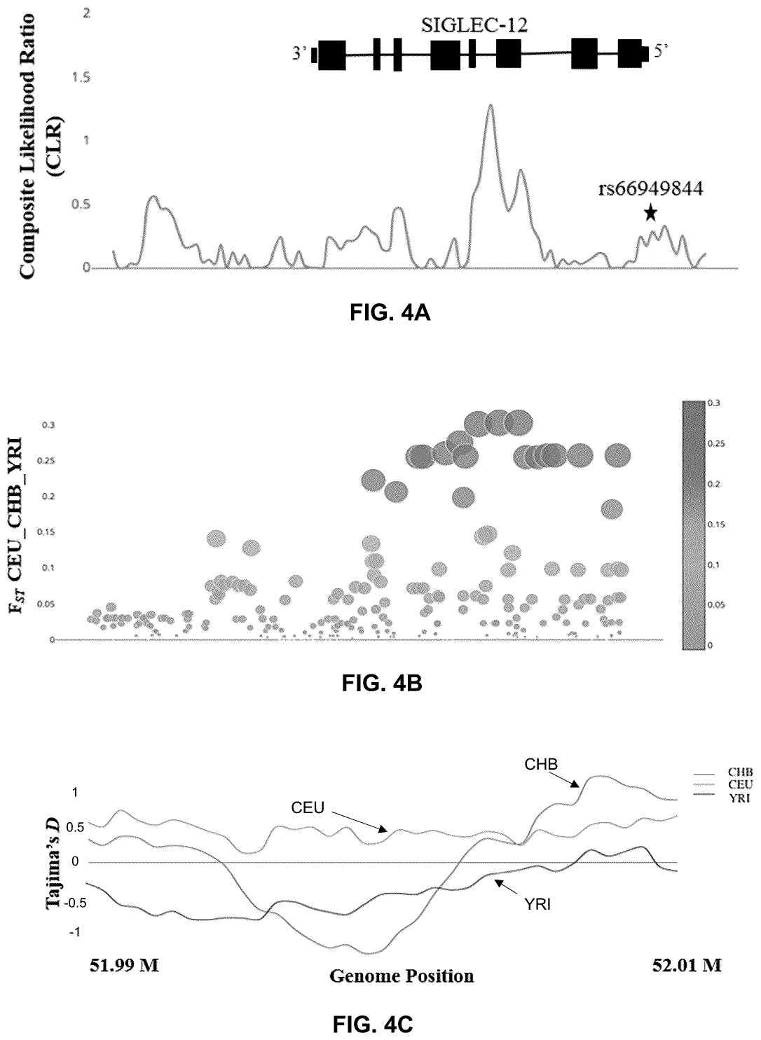

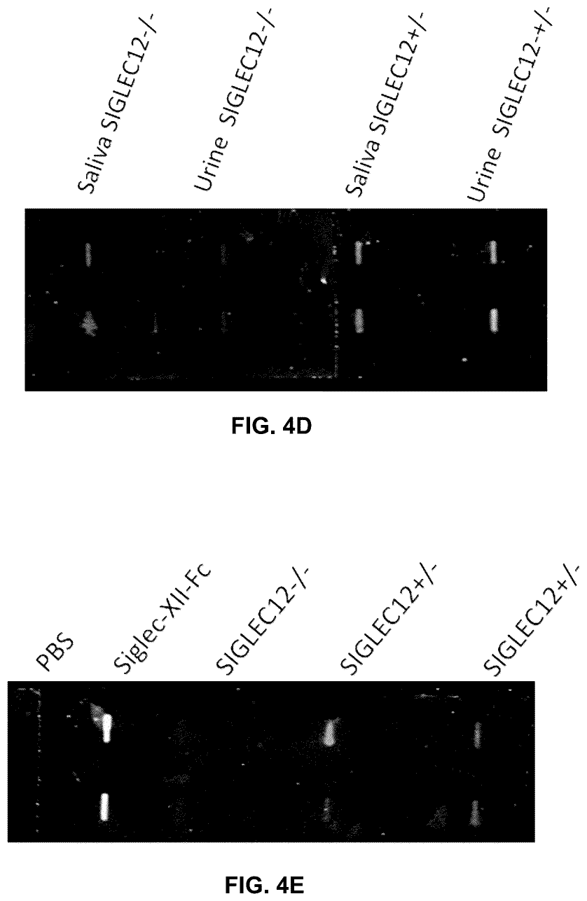

Earlier work suggested that this locus might be undergoing selection favoring the null state (4). The 1000 Genomes database was examined for further signatures of selection in and around the SIGLEC12 locus. Evidence of a soft “selective sweep” in the overall human population was found (FIG. 4A). Additionally, the common frame-shift mutation (rs66949844) was present adjacent to this area. Population differentiation was also estimated among three human populations and a reduced level of differentiation “FST” (global) was observed (FIG. 4B). Furthermore, the site frequency spectrum estimated by Tajima's D, showed an excess of rare alleles and reduction in diversity in human populations (FIG. 4C); especially in YRI (African ancestry population). Individually, none of these signals were very strong, but together, suggest selection for the null state (note that ongoing selection for a null state would favor inactivating mutations, which would tend to mask the usual signatures of a selective sweep).

### Example 7

### Dot Blot Analysis of Bladder Epithelial Cells for Detection of Siglec-XII Status

All the population studies above were handicapped by the fact that in addition to the common frameshift insertion mutation, other less common mutations that would nullify Siglec-XII expression were found. For example, another deleterious mutation (rs16982743) was observed at a global frequency of 18.6% that changes a glutamine to a stop codon (Q>*) at the 29th position (17). Thus, bi-allelic whole exome sequencing of SIGLEC12 genomic DNA would be required for rigorous population studies. Even this approach could be confounded by selection for non-coding mutations that suppress gene expression in a given allele with an intact open reading frame. To facilitate future population and cancer cohort studies, it would be useful to have a simple assay to rapidly detect all mutations abrogating expression, without the need to do whole exome sequencing. We took advantage of the fact that among normal epithelial tissues tested by IHC, Siglec-XII was expressed in bladder epithelium, kidney tubules and salivary gland ducts, and detected expression of Siglec-XII in cells isolated from saliva and urine. It was determined via buccal swab genomic analysis that the SIGLEC12 genomic status (SIGLEC12+/− or −/−) correlates with either Siglec-XII expression (+/−) or no expression (−/−). As expected, Siglec-XII expression in cells obtained from the urine of multiple healthy donors showed expression of Siglec-XII in the +/− genotypes and no expression in the Siglec-XII null genotypes. While there was significant background in samples from saliva, results from dot blot screening of urinary cells were very clean (a typical example is shown in FIG. 4E).

### Example 8

Expression of SIGLEC-XII in Human Carcinomas—Given the expression of Siglec-XII in certain epithelial cells, its expression was analyzed on human carcinomas (cancers of epithelial origin). Siglec-XII expression was indeed seen in many human prostate carcinoma specimens and also occasionally in breast carcinoma and in melanoma. In keeping with easily detectable expression in normal prostate epithelium, clear expression was found in prostate carcinomas (PCa). In the initial 50 PCa samples studied, there was a genotype to phenotype correlation, with no expression in samples in which both alleles were SIGLEC12P or frameshifted. Next, we looked for Siglec-XII expression in human breast and prostate carcinoma cell lines. Using flow cytometry, it was found that although MDaPCa2b and LnCAP (PCa) and MCF-7 (breast cancer) lines were positive, MDaMb231 (breast cancer) and PC-3 (PCa) showed no expression. As expected, the lack of expression correlated with the presence of the homozygous genomic SIGLEC12P allele, i.e. the frameshift.

Siglec-XII Expression in a Genotypically Null Prostate Carcinoma Cell Line Alters Expression of Multiple Genes Associated with Carcinoma Progression—Given the prominent expression of Siglec-XII in some human PCa and its absence in others with the homozygous SIGLEC12P, we wondered whether this had any functional consequences. As a first step toward addressing this question, we transfected the genotypically null PCa cell line, PC-3 with the intact human SIGLECl2 cDNA in the pcDNA3.1(−) expression vector, and established two lines with stable expression of cell surface Siglec-XII, using G418 selection (two empty vector transfected cell lines did not give a positive signal). We next studied total mRNA from both cell lines by microarray, looking for gene expression differences. We found limited but significant changes in gene expression between the SIGLEC12 and empty vector transfected cell lines. In all, 67 transcripts were identified as being down-regulated over the control upon Siglec-XII expression, at a false positive rate of 15%. Only MAP2K5 was up-regulated.

Interestingly, genes affected by Siglec-XII expression were involved in carcinoma progression such as matrix metalloproteinasel (MMT1), growth differentiation factor 15 (GDF-15/MIC-1), and RUNX2. A number of genes associated with cellular migration and adhesion such as CDHI, FGA, GDF15, IGFBP5, ITGB4, ITGB8, MMP1, RUNX2, S100A9, SDC2, TFF3, and TGFA were also down-regulated.

Anti-Siglec-XII Antibody-mediated Endocytotic Toxin Delivery into Prostate Cancer Cells—2500 stably transfected hSIGLEC12-PC3 cells were plated in 96-well plates in growth medium. Next day, different amounts of MabZAP (Advanced Targeting Systems, San Diego, CA) (0-200 ng) and anti-Siglec-12 antibodies 1130 or 276 (0-130 ng) were incubated together in cell growth medium for 30 min on ice. After aspirating media from the cells, the MabZAP-antibody mixture was added. Each combination of MabZAP-anti-Siglec-XII antibody was repeated in triplicate. The cells were further incubated for 3 days. The number of viable cells in each well was determined colorimetrically using the CellTiter 96® AQᵤₑₒᵤₛ One Solution cell proliferation assay (Promega).

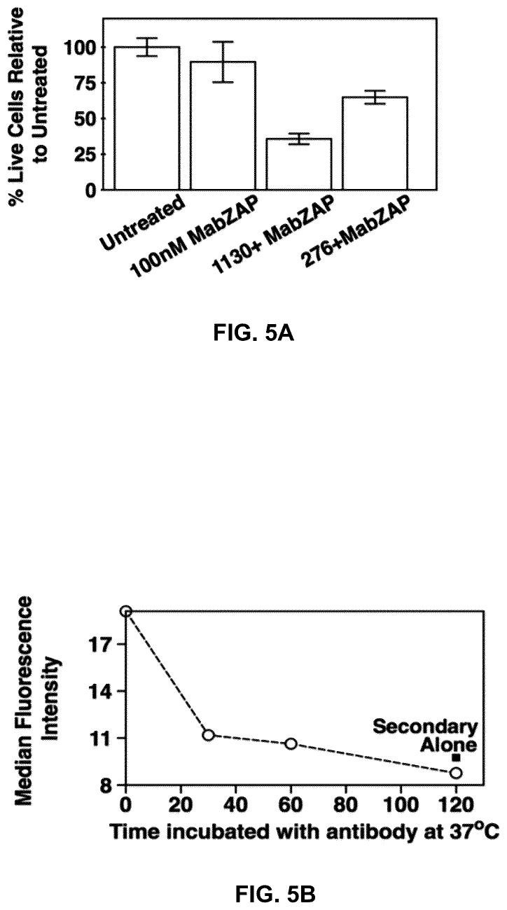

Monoclonal Antibody Binding Induces Internalization of Cell Surface Siglec-XII and Targeting of a Toxin into Human Carcinoma Cells—Previous studies have shown that Siglec-3, -5, and -9 undergo rapid internalization upon cross-linking with antibodies. If a toxin is attached to such antibodies, toxin internalization also occurs, and this results in cell death. We wanted to see whether Siglec-XII could be utilized to deliver a toxin into cells and thus cause cell death. For this, we used MabZAP, a goat anti-mouse antibody that was conjugated to the toxin saporin. Internalization of the Siglec would also deliver the mAb-MabZAP complex into the cell. To induce cell death, cells in a 96-well plate were incubated with primary antibody and MabZAP for 72 h. Following this, cell viability was determined. Wells with both antibody and the MabZAP had only 35% of cells alive. Wells with MabZAP alone had nearly as many cells alive, as wells with no treatment and wells with antibody alone showed some cell killing (FIG. 5A). Although we only performed toxicity studies on the stably transfected SIGLEC12 PC-3 cell line as a proof of principle experiment, we looked for Siglec-XII internalization in the prostate cell line MDaPCa2b (FIG. 5B), which natively expressed Siglec-XII, and saw that the Siglec-XII was completely internalized. Because plant and bacterial protein toxins are capable of killing cells at very low intracellular concentrations, we surmised that under the right conditions, cell death would occur, even with small amounts of surface Siglec-XII on carcinoma cells.

The following References are hereby incorporated by reference in their entireties.
1. T. Angata, N. M. Varki, A. Varki, J Biol Chem 276, 40282 (2001).
2. Z. Yu, C. M. Lai, M. Maoui, D. Banville, S. H. Shen, J Biol Chem 276, 23816 (2001).
3. N. Mitra et al., J Biol Chem 286, 23003 (2011).
4. B. Yngvadottir et al., Am J Hum Genet 84, 224 (2009).
5. F. Schwarz et al., Proc Natl Acad Sci USA 113, 74 (2016).
6. J. S. Brown, C. A. Aktipis, Philos Trans R Soc Lond B Biol Sci 370, (2015).
7. W. GC, Evolution 11, 398 (1957).
8. D. P. Croft, L. J. Brent, D. W. Franks, M. A. Cant, Trends Ecol Evol (2015).
9. K. Hawkes, J. E. Coxworth, Evol Anthropol 22, 294 (2013).
10. K. Hawkes, Proc Natl Acad Sci USA 113, 17 (2016).
11. E. Ungewitter, H. Scrable, Mech Ageing Dev 130, 10 (2009).
12. B. S. Bochner, N. Zimmermann, JAllergy Clin Immunol 135, 598 (2015).
13. O. J. Adams, M. A. Stanczak, S. von Gunten, H. Laubli, Glycobiology (2017).
14. A. Varki, R. L. Schnaar, P. R. Crocker, in I-Type Lectins, A. Varki et al., Eds. (Cold Spring Harbor Laboratory Press, Cold Spring Harbor (NY), 2017).
15. A. Varki, R. L. Schnaar, R. Schauer, in Sialic Acids and Other Nonulosonic Acids, A. Varki et al., Eds. (Cold Spring Harbor Laboratory Press, Cold Spring Harbor (NY), 2017).
16. G. Foussias et al., Biochem Biophys Res Commun 284, 887 (2001).
17. C. W. McDonough et al., Hypertension 62, 48 (2013).
18. M. S. Macauley, P. R. Crocker, J. C. Paulson, Nat Rev Immunol 14, 653 (2014).
19. N. M. Varki, A. Varki, Philos Trans R Soc Lond B Biol Sci 370, (2015).
20. J. A. Eastham et al., JAMA 289, 2695 (2003).
21. W. L. Beeson, P. K. Mills, R. L. Phillips, M. Andress, G. E. Fraser, Cancer 64, 570 (1989).
22. V. Heinemann et al., Lancet Oncol 15, 1065 (2014).
23. F. Loupakis et al., N Engl J Med 371, 1609 (2014).
24. L. Zhai et al., Clin Cancer Res 21, 5427 (2015).
25. N. Koide et al., Sci Rep 7, 43379 (2017).
26. W. D. Mahauad-Fernandez, K. A. DeMali, A. K. Olivier, C. M. Okeoma, Breast Cancer Res 16, 493 (2014).
27. W. F. Chiang et al., Oncogene 37, 116 (2018).
28. K. Stecker et al., Br J Cancer 104, 1426 (2011).
29. F. Wang et al., Cell Death Dis 5, e1133 (2014).
30. C. Ji et al., Oncol Res 26, 83 (2018).
31. J. Jen, Y. C. Wang, J Biomed Sci 23, 53 (2016).
32. D. R. Schrider, A. D. Kern, PLoS Genet 12, e1005928 (2016).
33. J. M. Akey, G. Zhang, K. Zhang, L. Jin, M. D. Shriver, Genome Res 12, 1805 (2002).
34. L. B. Barreiro, G. Laval, H. Quach, E. Patin, L. Quintana-Murci, Nat Genet 40, 340 (2008).
35. L. R. Bollu, A. Mazumdar, M. I. Savage, P. H. Brown, Clin Cancer Res 23, 2136 (2017).
36. R. Xu et al., Blood 106, 3142 (2005).
37. X. Zhou, J. Coad, B. Ducatman, Y. M. Agazie, Histopathology 53, 389 (2008).
38. F. Li, R. Zhang, S. Li, J. Liu, Int Immunopharmacol 47, 70 (2017).
39. L. Zhai et al., Cell Mol Immunol (2018).
40. G. C. Prendergast, W. P. Malachowski, J. B. DuHadaway, A. J. Muller, Cancer Res 77, 6795 (2017).
41. E. Raschperger et al., Exp Cell Res 312, 1566 (2006).
42. B. Vigl et al., Exp Cell Res 315, 336 (2009).
43. M. Reeh et al., Br J Cancer 109, 1848 (2013).
44. T. Okegawa et al., Cancer Res 60, 5031 (2000).
45. J. E. Coxworth, P. S. Kim, J. S. McQueen, K. Hawkes, Proc Natl Acad Sci USA 112, 11806 (2015).
46. L. M. Abegglen et al., JAMA 314, 1850 (2015).
47. J. K. Lamba et al., J Clin Oncol JC02016712513 (2017).
48. A. Dobin et al., Bioinformatics 29, 15 (2013).
49. Y. Liao, G. K. Smyth, W. Shi, Bioinformatics 30, 923 (2014).
50. M. I. Love, W. Huber, S. Anders, Genome Biol 15, 550 (2014).
51. H. Mi et al., Nucleic Acids Res 45, D183 (2017).
52. M. Pybus et al., Nucleic Acids Res 42, D903 (2014).
53. F. Tajima, Genetics 123, 585 (1989).
54. K. E. Holsinger, B. S. Weir, Nat Rev Genet 10, 639 (2009).
55. J. J. Vitti, S. R. Grossman, P. C. Sabeti, Annu Rev Genet 47, 97 (2013).
56. C. D. Huber, M. DeGiorgio, I. Hellmann, R. Nielsen, Mol Ecol 25, 142 (2016).
57. A. M. Aakhus, P. Stavem, T. Hovig, T. M. Pedersen, N. O. Solum, Br J Haematol 74, 320 (1990).
58. R. Nielsen et al., Genome Res 15, 1566 (2005).

Although the invention has been described with reference to the above disclosure, it will be understood that modifications and variations are encompassed within the spirit and scope of the invention. Accordingly, the invention is limited only by the following claims.

What is claimed is:

1. A method for detecting an increased risk of developing late stage progression of cancer in a subject undergoing treatment therefore, the method comprising:
(a) measuring the level of wild type Siglec-XII in a sample containing epithelial cells from the subject by contacting the sample with an antibody that specifically binds to wild type Siglec-XII and measuring the level of bound antibody in the sample to provide a measured level of bound antibody;
(b) comparing the measured level of the bound antibody to a reference level, wherein an elevated level of wild type Siglec-XII expression in the subject undergoing treatment for cancer is indicative of an increased risk in developing late stage progression of cancer.

2. The method of claim 1, further comprising administering an additional cancer therapy to the subject having an elevated level of wild type Siglec-XII expression.

3. The method of claim 2, wherein the additional cancer therapy comprises a complex comprising a monoclonal antibody and a toxin, and administration of the complex results in the death of cells expressing wild type Siglec-XII, thereby treating cancer in the subject.

4. The method of claim 1, wherein the sample is urine or saliva.

5. The method of claim 1, wherein the cancer is a carcinoma.

6. The method of claim 1, wherein the cancer is selected from skin cancer, colorectal cancer or prostate cancer.

7. The method of claim 1, wherein the antibody that specifically binds to wild type Siglec-XII comprises an anti-Siglec-XII monoclonal antibody 1130 or an anti-Siglec-XII monoclonal antibody 276.

8. The method of claim 1, wherein the epithelial cells comprise bladder epithelial cells.

9. The method of claim 1, further comprising measuring the expression level of one or more genes selected from the group consisting of IDO1, LCP1, BST2, CEACAM6, CXADR, TACSTD2, CTSF, and ZNF43, wherein elevated expression level of any one or more of IDO1, LCP1, BST2, and CEACAM6, and wherein decreased expression level of any one or more of CXADR, TACSTD2, CTSF, and ZNF43, as compared to expression level in a corresponding normal sample indicates late stage progression of the cancer in the subject and an additional cancer therapy is administered to the subject to treat the cancer.

10. The method of claim 9, wherein the levels of wild type Siglec-XII, IDO1, LCP1, BST2, CEACAM6, CXADR, TACSTD2, CTSF, and ZNF43 are measured with an immunoassay.

11. The method of claim 10, wherein the immunoassay is a sandwich assay, a fluoroimmunoassay, an immunofluorometric assay, an immunoradiometric assay, a luminescence assay or a chemiluminescence assay.

12. The method of claim 1, wherein the reference level is 0.

13. The method of claim 1, wherein the measured level of the bound antibody is greater than 0.

14. A method for detecting an increased risk of developing late stage progression of cancer in a subject, the method comprising:
(a) measuring the level of wild type Siglec-XII in a sample containing epithelial cells from the subject by contacting the sample with an antibody that specifically binds to wild type Siglec-XII and measuring the level of bound antibody in the sample to provide a measured level of bound antibody;
(b) comparing the measured level of the bound antibody to a reference level, wherein an elevated level of wild type Siglec-XII expression in the subject is indicative of an increased risk of developing late stage progression of cancer.

15. The method of claim 14, wherein the reference level is 0.

16. The method of claim 14, wherein the measured level of the bound antibody is greater than 0.

17. The method of claim 14, wherein the antibody that specifically binds to wild type Siglec-XII comprises an anti-Siglec-XII monoclonal antibody 1130 or an anti-Siglec-XII monoclonal antibody 276.

18. The method of claim 14, further comprising measuring the expression level of one or more genes selected from the group consisting of IDO1, LCP1, BST2, CEACAM6, CXADR, TACSTD2, CTSF, and ZNF43, wherein elevated expression level of any one or more of IDO1, LCP1, BST2, and CEACAM6, and wherein decreased expression level of any one or more of CXADR, TACSTD2, CTSF, and ZNF43, as compared to expression level in a corresponding normal sample indicates late stage progression of the cancer in the subject and an additional cancer therapy is administered to the subject to treat the cancer.

19. The method of claim 18, wherein the levels of wild type Siglec-XII, IDO1, LCP1, BST2, CEACAM6, CXADR, TACSTD2, CTSF, and ZNF43 are measured with an immunoassay.

20. The method of claim 19, wherein the immunoassay is a sandwich assay, a fluoroimmunoassay, an immunofluorometric assay, an immunoradiometric assay, a luminescence assay or a chemiluminescence assay.


SEQUENCE LISTING (20 sequences)

SEQ ID NO: 1 - protein, 595 aa, Homo sapiens
MLLLLLLLPPLLCGRVGAKEQKDYLLTMQKSVTVQEGLCVSVLCSFSYPQNGWTASDPVHGYWFRAGDHVSRNIPVATNNPARAVQEETRDRFHLLGDPQNKDCTLSIRDTRESDAGTYVFCVERGNMKWNYKYDQLSVNVTASQDLLSRYRLEVPESVTVQEGLCVSVPCSVLYPHYNWTASSPVYGSWFKEGADIPWDIPVATNTPSGKVQEDTHGRFLLLGDPQTNNCSLSIRDARKGDSGKYYFQVERGSRKWNYIYDKLSVHVTALTHMPTFSIPGTLESGHPRNLTCSVPWACEQGTPPTITWMGASVSSLDPTITRSSMLSLIPQPQDHGTSLTCQVTLPGAGVTMTRAVRLNISYPPQNLTMTVFQGDGTASTTLRNGSALSVLEGQSLHLVCAVDSNPPARLSWTWGSLTLSPSQSSNLGVLELPRVHVKDEGEFTCRAQNPLGSQHISLSLSLQNEYTGKMRPISGVTLGAFGGAGATALVFLYFCIIFVVVRSCRKKSARPAVGVGDTGMEDANAVRGSASQGPLIESPADDSPPHHAPPALATPSPEEGEIQYASLSFHKARPQYPQEQEAIGYEYSEINIPK

SEQ ID NO: 2 - DNA, 1000 bp, Homo sapiens
  source 1..1000: mol_type = unassigned DNA
atgctactgctgctgctactgctgccacccctgctctgtgggagagtgggggctaaggaacagaaggattacctgctgacaatgcagaagtccgtgacggtgcaggagggcctgtgtgtctctgtgctttgctccttctcctacccccaaaatggctggactgcctccgatccagttcatggctactggttccgggcaggggaccatgtaagccggaacattccagtggccacaaacaacccagctcgagcagtgcaggaggagactcgggaccgattccacctccttggggacccacagaacaaggattgtaccctgagcatcagagacaccagagagagtgatgcagggacatacgtcttttgtgtagagagaggaaatatgaaatggaattataaatatgaccagctctctgtgaatgtgacagcgtcccaggacctactgtcaagatacaggctggaggtgccagagtcggtgactgtgcaggagggtctgtgtgtctctgtgccctgcagtgtcctttacccccattacaactggactgcctctagccctgtttatggatcctggttcaaggaaggggccgatataccatgggatattccagtggccacaaacaccccaagtggaaaagtgcaagaggatacccacggtcgattcctcctccttggggacccacagaccaacaactgctccctgagcatcagagatgccaggaagggggattcagggaagtactacttccaggtggagagaggaagcaggaaatggaactacatatatgacaagctctctgtgcatgtgacagccctgactcacatgcccaccttctccatcccggggaccctggagtctggccaccccaggaacctgacctgctctgtgccctgggcctgtgaacaggggacgccccccacgatcacctggatgggggcctccgtgtcctccctggaccccactatcactcgctcctcgatgctcagcctcatcccacagcccc

SEQ ID NO: 3 - protein, 477 aa, Homo sapiens
MLLPLLWANEERDSGGWADPRFSTASQDLLSRYRLEVPESVTVQEGLCVSVPCSVLYPHYNWTASSPVYGSWFKEGADIPWDIPVATNTPSGKVQEDTHGRFLLLGDPQTNNCSLSIRDARKGDSGKYYFQVERGSRKWNYIYDKLSVHVTALTHMPTFSIPGTLESGHPRNLTCSVPWACEQGTPPTITWMGASVSSLDPTITRSSMLSLIPQPQDHGTSLTCQVTLPGAGVTMTRAVRLNISYPPQNLTMTVFQGDGTASTTLRNGSALSVLEGQSLHLVCAVDSNPPARLSWTWGSLTLSPSQSSNLGVLELPRVHVKDEGEFTCRAQNPLGSQHISLSLSLQNEYTGKMRPISGVTLGAFGGAGATALVFLYFCIIFVVVRSCRKKSARPAVGVGDTGMEDANAVRGSASQGPLIESPADDSPPHHAPPALATPSPEEGEIQYASLSFHKARPQYPQEQEAIGYEYSEINIPK

SEQ ID NO: 4 - DNA, 1000 bp, Homo sapiens
  source 1..1000: mol_type = unassigned DNA
atgctgctgcccctgctatgggcaaatgaagagagggacagtgggggctgggctgaccctcgtttctccacagcgtcccaggacctactgtcaagatacaggctggaggtgccagagtcggtgactgtgcaggagggtctgtgtgtctctgtgccctgcagtgtcctttacccccattacaactggactgcctctagccctgtttatggatcctggttcaaggaaggggccgatataccatgggatattccagtggccacaaacaccccaagtggaaaagtgcaagaggatacccacggtcgattcctcctccttggggacccacagaccaacaactgctccctgagcatcagagatgccaggaagggggattcagggaagtactacttccaggtggagagaggaagcaggaaatggaactacatatatgacaagctctctgtgcatgtgacagccctgactcacatgcccaccttctccatcccggggaccctggagtctggccaccccaggaacctgacctgctctgtgccctgggcctgtgaacaggggacgccccccacgatcacctggatgggggcctccgtgtcctccctggaccccactatcactcgctcctcgatgctcagcctcatcccacagccccaggaccatggcaccagcctcacctgtcaggtgaccttgcctggggccggcgtgaccatgaccagggctgtccgactcaacatatcctatcctcctcagaacttgaccatgactgtcttccaaggagatggcacagcatccacaaccttgaggaatggctcggccctttcagtcctggagggccagtccctgcaccttgtctgtgctgtcgacagcaatccccctgccaggctgagctggacctgggggagcctgaccctgagcccctcacagtcctcgaaccttggggtgctggagctgcctcgagtgcatgtgaaggatgaaggggaattcacctgccgagctcagaaccctc

SEQ ID NO: 5 - protein, 597 aa, Pan troglodytes
MLLLLLLLLLPPLLCGRVGAKEQKDYLLTMQKSVTVQEGLCVSVLCSFSYPQNGWTDSDPVHGYWFRAGDHVSRNVPVATNNPARAVQEETRDRFHLLGDPQNKDCTLSIRDTRESDAGTYVFRVERGNMKWNYKYDQLSVNVTASQDLLSRYRLEVPESVTVQEGLCVSVPCSVLYPHCNWTASSPVYGSWFKEGADIPCDIPVATNTPSGKVQEDTQGRFLLLGDPQTNNCSLSIRDARKGDSGKYYFQVERGSRKWNYIYDKLSVHVTALTHLPTFSIPGTLESGHPRNLTCSVPWACEQGTPPTITWMGASVSSLEPTISRSSMLSLIPKPQDHGTSLTCQVTLPGAGVTTTRAVRLNISYPPQNLTMTVFQGDGTASTTLRNGSALSVLEGQSLHLVCAVDSNPPARLSWTWGSLTLSPSQSSNLGVLELPRVHVKDEGEFTCRAQNPLGSQHISLSLSLQNEYTGKMRPISGVTLGAVGGAGATALVFLSFCIIFVVVRSCRKKSARPAVGVGDTGMEDTNAVRGSASQGPLIESPADDSPPHHAPPALATPFPEEGEIQYASLSFHKARPQYPQEQEAIGYEYSEINILK

SEQ ID NO: 6 - DNA, 1000 bp, Pan troglodytes
  source 1..1000: mol_type = unassigned DNA
atgctactgctgctgctactgctactgctgccacccctgctctgtgggagagtgggggctaaggaacagaaggattacctgctgacaatgcagaagtccgtgacggtgcaggagggcctgtgtgtctctgtgctttgctccttctcctacccccaaaatggctggactgactccgatccagttcatggctactggttccgggcaggggaccatgtaagccggaacgttccagtggccacaaacaacccagctcgagcagtgcaggaggagactcgggaccgattccacctccttggggacccacagaacaaggattgtaccctgagcatcagagacaccagagagagtgatgcagggacatacgtctttcgtgtagagagaggaaatatgaaatggaattataaatatgaccagctctctgtgaatgtgacagcgtcccaggacctactgtcaagatacaggctggaggtgccagagtcggtgactgtgcaggagggtctgtgtgtctctgtgccctgcagtgtcctttacccccattgcaactggactgcctctagccctgtttatggatcctggttcaaggaaggggccgatataccatgcgatattccagtggccacaaacaccccaagtggaaaagtgcaagaggatacccagggtcgattcctcctccttggggacccacagaccaacaactgctccctgagcatcagagatgccaggaagggggattcagggaagtactacttccaggtggagagaggaagcaggaaatggaactacatatatgacaagctctctgtgcatgtgacagccctgactcacttgcccaccttctccatcccggggaccctggagtctggccaccccaggaacctgacctgctctgtgccctgggcctgtgaacaggggacgccccccacaatcacctggatgggggcctccgtgtcctccctggaacccactatctcccgctcctcgatgctcagcctcatcccaa

SEQ ID NO: 7 - protein, 403 aa, Homo sapiens [sequence omitted]

SEQ ID NO: 8 - protein, 627 aa, Homo sapiens
MARGSVSDEEMMELREAFAKVDTDGNGYISFNELNDLFKAACLPLPGYRVREITENLMATGDLDQDGRISFDEFIKIFHGLKSTDVAKTFRKAINKKEGICAIGGTSEQSSVGTQHSYSEEEKYAFVNWINKALENDPDCRHVIPMNPNTNDLFNAVGDGIVLCKMINLSVPDTIDERTINKKKLTPFTIQENLNLALNSASAIGCHVVNIGAEDLKEGKPYLVLGLLWQVIKIGLFADIELSRNEALIALLREGESLEDLMKLSPEELLLRWANYHLENAGCNKIGNFSTDIKDSKAYYHLLEQVAPKGDEEGVPAVVIDMSGLREKDDIQRAECMLQQAERLGCRQFVTATDVVRGNPKLNLAFIANLFNRYPALHKPENQDIDWGALEGETREERTFRNWMNSLGVNPRVNHLYSDLSDALVIFQLYEKIKVPVDWNRVNKPPYPKLGGNMKKLENCNYAVELGKNQAKFSLVGIGGQDLNEGNRTLTLALIWQLMRRYTLNILEEIGGGQKVNDDIIVNWVNETLREAKKSSSISSFKDPKISTSLPVLDLIDAIQPGSINYDLLKTENLNDDEKLNNAKYAISMARKIGARVYALPEDLVEVNPKMVMTVFACLMGKGMKRV

SEQ ID NO: 9 - protein, 180 aa, Homo sapiens
MASTSYDYCRVPMEDGDKRCKLLLGIGILVLLIIVILGVPLIIFTIKANSEACRDGLRAVMECRNVTHLLQQELTEAQKGFQDVEAQAATCNHTVMALMASLDAEKAQGQKKVEELEGEITTLNHKLQDASAEVERLRRENQVLSVRIADKKYYPSSQDSSSAAAPQLLIVLLGLSALLQ

SEQ ID NO: 10 - protein, 526 aa, Homo sapiens
MGHLSAPLHRVRVPWQGLLLTASLLTFWNPPTTAQLTTESMPFNVAEGKEVLLLVHNLPQQLFGYSWYKGERVDGNRQIVGYAIGTQQATPGPANSGRETIYPNASLLIQNVTQNDTGFYTLQVIKSDLVNEEATGQFHVYPELPKPSISSNNSNPVEDKDAVAFTCEPETQDTTYLWWINNQSLPVSPRLQLSNGNRTLTLLSVTRNDTGPYECEIQNPVSANRSDPVTLNVTYGPDTPTISPSDTYYRPGANLSLSCYAASNPPAQYSWLINGTFQQSTQELFIPNITVNNSGSYTCHANNSVTGCNRTTVKTIIVTELSPVVAKPQIKASKTTVTGDKDSVNLTCSTNDTGISIRWFFKNQSLPSSERMKLSQGNTTLSINPVKREDAGTYWCEVFNPISKNQSDPIMLNVNYNALPQENGLSPGAIAGIVIGVVALVALIAVALACFLHFGKTGRASDQRDLTEHKPSVSNHTQDHSNDPPNKMNEVTYSTLNFEAQQPTQPTSASPSLTATEIIYSEVKKQ

SEQ ID NO: 11 - protein, 365 aa, Homo sapiens
MALLLCFVLLCGVVDFARSLSITTPEEMIEKAKGETAYLPCKFTLSPEDQGPLDIEWLISPADNQKVDQVIILYSGDKIYDDYYPDLKGRVHFTSNDLKSGDASINVTNLQLSDIGTYQCKVKKAPGVANKKIHLVVLVKPSGARCYVDGSEEIGSDFKIKCEPKEGSLPLQYEWQKLSDSQKMPTSWLAEMTSSVISVKNASSEYSGTYSCTVRNRVGSDQCLLRLNVVPPSNKAGLIAGAIIGTLLALALIGLIIFCCRKKRREEKYEKEVHHDIREDVPPPKSRTSTARSYIGSNHSSLGSMSPSNMEGYSKTQYNQVPSEDFERTPQSPTLPPAKVAAPNLSRMGAIPVMIPAQSKDGSIV

SEQ ID NO: 12 - protein, 323 aa, Homo sapiens
MARGPGLAPPPLRLPLLLLVLAAVTGHTAAQDNCTCPTNKMTVCSPDGPGGRCQCRALGSGMAVDCSTLTSKCLLLKARMSAPKNARTLVRPSEHALVDNDGLYDPDCDPEGRFKARQCNQTSVCWCVNSVGVRRTDKGDLSLRCDELVRTHHILIDLRHRPTAGAFNHSDLDAELRRLFRERYRLHPKFVAAVHYEQPTIQIELRQNTSQKAAGDVDIGDAAYYFERDIKGESLFQGRGGLDLRVRGEPLQVERTLIYYLDEIPPKFSMKRLTAGLIAVIVVVVVALVAGMAVLVITNRRKSGKYKKVEIKELGELRKEPSL

SEQ ID NO: 13 - protein, 484 aa, Homo sapiens [sequence omitted]

SEQ ID NO: 14 - protein, 809 aa, Homo sapiens
MGPLTFMDVAIEFCLEEWQCLDIAQQNLYRNVMLENYRNLVFLGIAVSKPDLITCLEQEKEPWEPMRRHEMVAKPPVMCSHFTQDFWPEQHIKDPFQKATLRRYKNCEHKNVHLKKDHKSVDECKVHRGGYNGFNQCLPATQSKIFLFDKCVKAFHKFSNSNRHKISHTEKKLFKCKECGKSFCMLPHLAQHKIIHTRVNFCKCEKCGKAFNCPSIITKHKRINTGEKPYTCEECGKVFNWSSRLTTHKKNYTRYKLYKCEECGKAFNKSSILTTHKIIRTGEKFYKCKECAKAFNQSSNLTEHKKIHPGEKPYKCEECGKAFNWPSTLTKHKRIHTGEKPYTCEECGKAFNQFSNLTTHKRIHTAEKFYKCTECGEAFSRSSNLTKHKKIHTEKKPYKCEECGKAFKWSSKLTEHKLTHTGEKPYKCEECGKAFNWPSTLTKHNRIHTGEKPYKCEVCGKAFNQFSNLTTHKRIHTAEKPYKCEECGKAFSRSSNLTKHKKIHIEKKPYKCEECGKAFKWSSKLTEHKITHTGEKPYKCEECGKAFNHFSILTKHKRIHTGEKPYKCEECGKAFTQSSNLTTHKKIHTGEKFYKCEECGKAFTQSSNLTTHKKIHTGGKPYKCEECGKAFNQFSTLTKHKIIHTEEKPYKCEECGKAFKWSSTLTKHKIIHTGEKPYKCEECGKAFKLSSTLSTHKIIHTGEKPYKCEKCGKAFNRSSNLIEHKKIHTGEQPYKCEECGKAFNYSSHLNTHKRIHTKEQPYKCKECGKAFNQYSNLTTHNKIHTGEKLYKPEDVTVILTTPQTFSNIK

SEQ ID NO: 15 - DNA, 27 bp, synthetic construct
  misc_feature 1..27: note = synthetic construct
  source 1..27: mol_type = other DNA
caatgcagaagtccgtgacggtgcagg

SEQ ID NO: 16 - DNA, 26 bp, synthetic construct
  misc_feature 1..26: note = synthetic construct
  source 1..26: mol_type = other DNA
aggatcaggaggggcatccaaggtgc

SEQ ID NO: 17 - DNA, 22 bp, synthetic construct
  misc_feature 1..22: note = synthetic construct
  source 1..22: mol_type = other DNA
ctctctctggtgtctctgatgc

SEQ ID NO: 18 - DNA, 21 bp, synthetic construct
  misc_feature 1..21: note = synthetic construct
  source 1..21: mol_type = other DNA
acccctgctctgtgggagagt

SEQ ID NO: 19 - DNA, 26 bp, synthetic construct
  misc_feature 1..26: note = synthetic construct
  source 1..26: mol_type = other DNA
aggatcaggaggggcatccaaggtgc

SEQ ID NO: 20 - DNA, 22 bp, synthetic construct
  misc_feature 1..22: note = synthetic construct
  source 1..22: mol_type = other DNA
ctctctctggtgtctctgatgc